(19) United States
(12) Patent Application Publication
(10) Pub. No.: US 2010/0120651 A1
(43) Pub. Date: May 13, 2010

(54) ENZYME FABRIC CARE TABLETS FOR CONSUMERS AND METHODS

(75) Inventors: Douglas A. Dale, Pacifica, CA (US); David A. Estell, San Francisco, CA (US); Beth Fryksdale, San Jose, CA (US); Alfred L. Gaertner, San Bruno, CA (US); Kenneth F. Herfert, Belmont, CA (US); Philippe Lavielle, Maisons Laffitte (FR); Janet C. Naab, North Chili, NY (US); Thomas Pekich, Loudon, TN (US)

Correspondence Address: DANISCO US INC.;ATTENTION: LEGAL DEPARTMENT, 925 PAGE MILL ROAD, PALO ALTO, CA, 94304, US

(73) Assignee: GENENCOR INTERNATIONAL, INC., Palo Alto, CA (US)

(21) Appl. No.: 11/988,467

(22) PCT Filed: Jul. 11, 2006
(86) PCT No.: PCT/US2006/026705

§ 371 Date: Dec. 21, 2009

Related U.S. Application Data
(60) Provisional application No. 60/698,386, filed on Jul. 11, 2005.

(51) Int. Cl. C11D 3/386; C12N 9/00; C11D 17/00; C12N 9/42; C12N 9/16; C12N 9/20; C12N 9/48; C12N 9/02; C12N 9/26

(52) U.S. Cl. 510/224; 435/183; 510/276; 510/320; 435/209; 435/196; 435/198; 435/212; 435/189; 435/201

(57) ABSTRACT

Single-dose enzyme tablets for direct sale to consumers are provided for boosting the performance of automatic laundry and dish washing operations using conventional detergents. The tablets provide a wash performance that is better than or at least equal to the wash performance of the detergents alone even when the time and or temperature of the wash cycle is decreased. The tablets may be provided in kits and the enzymes are selected to treat a variety of fabric stains as well as to provide fabric care benefits.

### RELATED APPLICATIONS

This application claims priority from U.S. Provisional Application No. 60/698,386, filed Jul. 11, 2005, which is hereby incorporated by reference in its entirety.

### FIELD OF THE INVENTION

This invention relates to enzymes for direct sale to consumers. More specifically, the invention relates to enzyme tablets for use by consumers, the tablets enhancing and/or supplementing the performance of commercially available fabric and dish care products and providing a cleaning benefit. Yet more specifically, the invention relates to a variety of enzyme tablets with different enzymes and enzyme combinations for selection by a consumer to provide enhanced cleaning, and/or to provide stain specific cleaning, and/or to provide fabric enhancements, and/or to provide a cleaning benefit when added by the consumer to cleaning and fabric care procedures using commercially available cleaning and fabric care products.

### BACKGROUND OF THE INVENTION

The cleaning industry, particularly the laundry and dish detergent industry, provides products with varying sophistication and performance qualities based, at least in part, upon production costs. For example, the cleaning industry sells generic “store brand” economy grade products as well as premium performance products.

The wash cycle settings for automatic washing machines generally have somewhat similar parameters of wash time and wash temperature. For instance, “Regular” wash cycle settings in the United States are generally between about 12 minutes to about 18 minutes at about 33° C. to about 43° C. (“Warm” setting), while those in European washing machines generally are between about 100 minutes to about 140 minutes (“Normal” setting) at about 40° C. to about 60° C. Wash settings in Asia generally are about 15 minutes at about 25° C. Decreases in the time and/or wash temperature of these “Regular” and “Normal” wash cycles generally leads to decreased wash performance.

Premium performance products generally include one or more enzymes that remove different stain components. These premium products must be designed and formulated to protect the enzymes during storage of the product, for instance by preventing cross-reactivity with other ingredients, to maintain stability and performance of the enzymes when stored in the products, and to minimize the possibility of allergic reactions from the enzymes, which are proteins. Changes in the cleaning products may significantly affect the enzyme performance and stability thereby requiring additional changes to the enzyme formulation, or to the enzyme itself, to overcome possible negative effects on the enzymes.

The industry uses enzyme granules that are distributed throughout the cleaning product, and the granules must have protective ingredients surrounding the enzymes to combat stability and sensitization issues. Enzyme manufactures also produce modified or engineered enzymes with properties that can make the enzymes more stable when stored in cleaning products, including liquid cleaning products, but such modified and engineered enzymes continue to require enzyme formulations or enzyme modifications that provide at least some protection for the enzyme when stored in the cleaning product. The performance of enzymes stored in cleaning products does tend to decline over time, and this problem is particularly severe in liquid cleaning products. Storage effects on enzymes increase costs to both cleaning product manufacturers and enzyme component suppliers, as formulating the enzymes to survive storage in cleaning products is expensive and challenging.

Economy grade cleaning products typically do not include enzymes, and such products lack cleaning performance when compared with premium performance products that contain enzymes.

It is desirable to use enzymes in combination with surfactants and/or bleaching agents found in laundry and dish detergents to improve cleaning. Unfortunately, enzymes are deactivated in the presence of the bleach ingredients and also in the presence of surfactants and detergents during storage. Thus, commonly commercially available liquid detergents do not contain bleach. Examples of bleaching agents include inorganic perhydrates, such as sodium perborate or sodium percarbonate, organic peracids, and chlorine-containing bleaches. The bleach is normally present in powder detergents from about 5% to about 40% by weight. Additionally, bleach containing detergents often contain one or more bleach activators, such as N,N,N′,N′-tetraacetylethylendiamine (TAED), pentaacetylglucose (PAG) and tetraacetyl (glycol uril) (TAGU). The bleach activator can be present in the detergent in an amount from about 0.1 to about 20% by weight. Examples of surfactants include nonionic, anionic, ampholytic, zwitterionic, or cationic surfactants. Common ingredients in laundry detergents are anionic surfactants such as linear alkyl benzene sulfonate (LAS) and non-ionic surfactants such as alcohol ethoxysulfate/alcohol ethoxylate (AES/AE). “Laundry detergent compatibility of the alkaline protease from Bacillus cereus”, Banika, Rathindra Mohan and Prakash, Monika, Microbiological Research, Volume 159, Issue 2, pp 135-140 (2004), discussing factors that destabilize laundry enzymes including bleach and surfactants. “Formulation of Detergent Enzymes”, Becker et al., Enzymes in Detergency, Jan H van Ee and Onno Misset, published by Erik J Baas, NY Marcel Dekker (1997), which describes methods for stabilizing enzymes in detergents. It is expensive and technically challenging to deliver enzymes for use with laundry and dish detergents having surfactants and/or bleaching agents and bleach activators.

EP0481547A1 discloses a dishwasher detergent tablet comprising a surfactant, enzymes and chlorine bleaches having at least three layers to separate “incompatible ingredients such as an enzyme and a chlorine bleach.” U.S. Pat. No. 6,413,928 describes a process for the preparation of a multi-phase detergent tablet that has a gelatinous portion. The tablet physically separates the enzyme from the bleaching agents and surfactant. A commercially available product said to have both enzymes and bleaching agents is available in tablet form and is known as Vanish Action Ball from Reckitt Benckiser.

For economy grade laundry or dish detergents, the cost structure often does not allow for the use of enzymes and does not allow for the use of complex, costly formulations that would separate reactive agents from enzymes.

The present invention provides a cost effective way to deliver enzymes directly to the consumer without encountering enzyme stability problems because the enzyme tablet of the present invention does not contain bleaching agents or bleach activators or the surfactants typically found in laundry and dish detergents.

There is a need in the industry for selectable single-dose, solid enzyme products that may be sold directly to the consumer who selects and adds the enzyme product as needed during a cleaning procedure using existing cleaning formulations, particularly economy cleaning formulations without enzymes and liquid cleaning formulations.

There is also a need in the industry for products that enable reduced energy consumption during wash cycles.

### SUMMARY OF THE INVENTION

The present invention relates to a variety of solid enzyme tablets for direct sale to consumers who may select an enzyme tablet and add the tablet to a cleaning and/or fabric care operation depending upon the particular cleaning need.

In a first embodiment an enzyme tablet provides a cleaning benefit as a single-dose additive to the wash cycle of an automatic laundry or dish washer, the wash cycle having a laundry or dish detergent therein; the enzyme tablet comprising one or more enzymes, with the proviso that the tablet is substantially without surfactants and bleaching agents; the cleaning benefit is selected from a decreased temperature of a wash cycle, a decreased time of a wash cycle, increased wash performance, and combinations thereof.

In a second embodiment an enzyme kit is provided for consumers, the kit comprising at least one enzyme containing tablet and packaging material, the at least one enzyme tablet providing a cleaning benefit as a single-dose additive to a laundry or dish washing cycle having a conventional laundry or dish detergent.

In a third embodiment a method is provided for boosting the wash performance of a conventional laundry or dish detergent in a laundry or dish washer; the method comprising: selecting at least one enzyme for laundry or dish detergent applications; combining the at least one enzyme with at least binders or fillers; manufacturing tablets from the combined enzyme and binders or fillers; and packaging the tablets for sale to consumers with instructions for use.

In a fourth embodiment a method is provided to provide a cleaning benefit in a wash cycle in laundry and dish washing applications, the method comprising: selecting on a laundry or dish washing machine a decreased temperature and/or a decreased time for a wash cycle; adding a laundry or dish washing compound and an enzyme tablet to the selected wash cycle; the enzyme tablet providing a cleaning benefit to the laundry or dish washing cycle, the cleaning benefit selected from decreased temperature of a wash cycle, decreased time of a wash cycle, increased wash performance, and combinations thereof.

In a fifth embodiment an enzyme tablet additive to the wash cycle of an automatic laundry or dish washer is provided, the wash cycle having a laundry or dish detergent therein; the enzyme tablet a single-dose additive comprising an enzyme, with the proviso that the tablet is substantially without surfactants and bleaching agents; the enzyme tablet enabling reduction of a time of a European wash cycle by at least 50% and/or reduction of a temperature of the wash cycle by at least 30%.

In kit embodiments of the invention a variety of solid enzyme tablets are provided as a cleaning kit with the variety including different enzymes, and/or enzyme combinations, designed to boost the performance of a cleaning compound and/or remove specific stains. Packages of a stain-specific enzyme tablets are provided.

In the embodiments of the invention an enzyme tablet may be provided for treating fabric with grass stains, the tablet having one or more enzymes selected from protease, cell wall degrading enzymes such as cellulase, hemicellulase, pectinase, and pectate lyases.

In the embodiments of the invention an enzyme tablet may be provided for treating fabric with blood stains, the tablet having one or more enzymes selected from protease, lipase and phospholipase.

In the embodiments of the invention an enzyme tablet may be provided for treating fabric with food stains, the tablet having one or more enzymes selected from protease, amylase, pectinase, pectate lyase, hemicellulase, mannanase, lipase, and oxidases.

In the embodiments of the invention an enzyme tablet may be provided for treating fabrics stained with body oils (sebum) and sometimes referred to as dingy fabrics, the tablet having one or more enzymes selected from protease, lipase, phospholipase, and oxidases.

In the embodiments of the invention an enzyme tablet may be provided for treating fabric with grass, blood, and food stains, the tablet having one or more enzymes selected from protease, amylase, cellulase, pectinase, pectate lyase, hemicellulase, mannanase, lipase, phospholipase, and oxidases.

In the embodiments of the invention an enzyme tablet may be provided for providing laundry bleaching benefits, the tablet having one or more enzymes selected from peroxidase, oxidase, and laccase. Additionally, the bleaching benefit may be provided by adding enzyme-substrate combinations, such as glucose oxidase and glucose, laccase and a mediator, and a lipase or esterase, ester and peroxide generating compound.

In the embodiments of the invention an enzyme tablet may be provided for addition to an automatic dishwashing procedure to add a cleaning benefit, the tablet having one or more enzymes selected from protease, amylase, pectinase, pectate lyase, hemicellulase, mannanase, lipase, phospholipase, and oxidase.

In the embodiments of the invention an enzyme tablet may be provided for use with boron free detergents, the tablet providing treatment for fabrics with stains and having one or more enzymes selected from protease, amylase, pectinase, pectate lyase, hemicellulase, mannanase, lipase, phospholipase, and oxidase.

In the embodiments of the invention an enzyme tablet may be provided for treating and conditioning cotton and polyester fabrics, the tablet having one or more enzymes selected from cellulase, cutinase, and esterase (polyesterase).

The enzyme tablets may be packaged separately by application, and they may be provided as kits that contain a variety of enzyme tablets for varying applications. The tablets may be color-coded to identify application.

### DESCRIPTION OF THE DRAWINGS

FIG. 1 is a graph showing the soil removal performance of Tandil™ detergent with and without a tablet of the present invention and with a commercially available laundry booster known as Vanish Action Ball™.

FIG. 2 is a graph showing the soil removal performance of IEC detergent (without bleach and enzymes) with and without a tablet of the present invention and a with a commercially available laundry booster known as Vanish Action Ball™.

FIG. 3 is a graph showing the soil removal performance of IEC detergent (with bleach and without enzymes) with and without a tablet of the present invention and a with a commercially available laundry booster known as Vanish Action Ball™.

FIG. 4 is a graph showing the soil removal performance of Tide® detergent with and without added protease tablets of the present invention.

FIG. 5 is a graph showing the soil removal performance of Purex® detergent with and without added protease tablets of the present invention.

FIG. 6 is a graph showing the soil removal performance of Nice™ detergent with and without added protease tablets of the present invention.

FIG. 7 is a graph showing the soil removal performance of Excel™ detergent with and without added protease tablets of the present invention.

FIG. 8 is a photograph of an enzyme tablet of the present invention, the tablet is a 0.95 cm by 2.5 cm capsule shaped tablet with beveled edges.

FIG. 9 is a photograph of an enzyme tablet of the present invention, the tablet is 2 cm in diameter and is a spheroid shaped tablet.

FIG. 10 is a graph showing the storage stability of an enzyme tablet of the present invention made without bleaching agents, bleach activators, surfactants, and enzyme stabilizers.

FIG. 11 is a graph showing the storage stability of an enzyme tablet made with bleaching agents and bleach activators and without enzyme stabilizers.

### DETAILED DESCRIPTION OF THE INVENTION

The present invention relates to a variety of solid enzyme tablets for direct sale to consumers who may select a solid enzyme tablet and add the tablet to a cleaning and/or fabric care operation to provide a cleaning benefit depending upon the particular cleaning need.

In a first embodiment of the invention a variety of solid enzyme tablets are provided as a cleaning kit with the variety including different enzymes, and/or enzyme combinations, designed to boost the performance of a cleaning compound and/or remove specific stains.

In a second embodiment of the invention packages of a stain-specific enzyme tablet are provided.

In a third embodiment of the invention an enzyme tablet is provided for treating fabric with grass stains, the tablet having one or more enzymes selected from protease, cell wall degrading enzymes such as cellulase, hemicellulase, pectinase, and pectate lyases.

In a fourth embodiment of the invention an enzyme tablet is provided for treating fabric with blood stains, the tablet having one or more enzymes selected from protease, lipase and phospholipase.

In a fifth embodiment of the invention an enzyme tablet is provided for treating fabric with food stains, the tablet having one or more enzymes selected from protease, amylase, pectinase, pectate lyase, hemicellulase, mannanase, lipase, and oxidases.

In a sixth embodiment of the invention an enzyme tablet is provided for treating fabric that is stained with body oils (sebum) and sometimes referred to as dingy fabric, the tablet having one or more enzymes selected from protease, lipase, phospholipase, and oxidases.

In a seventh embodiment of the invention an enzyme tablet is provided for treating fabric with grass, blood, and food stains, the tablet having one or more enzymes selected from protease, amylase, cellulase, pectinase, pectate lyase, hemicellulase, mannanase, lipase, phospholipase, and oxidases.

In an eighth embodiment of the invention an enzyme tablet is provided for addition to an automatic dishwashing procedure to add a cleaning benefit, the tablet having one or more enzymes selected from protease, amylase, pectinase, pectate lyase, hemicellulase, mannanase, lipase, phospholipase, and oxidase.

In a ninth embodiment of the invention an enzyme tablet is provided for use with boron free detergents, the tablet providing treatment for fabrics with stains and having one or more enzymes selected from protease, amylase, pectinase, pectate lyase, hemicellulase, mannanase, lipase, phospholipase, and oxidase.

In a tenth embodiment of the invention an enzyme tablet is provided for treating and conditioning cotton and polyester fabrics, the tablet having one or more enzymes selected from cellulase, cutinase, and esterase (polyesterase).

The above embodiments are not meant to be limiting, and it will be recognized by those skilled in the art that other enzymes and enzyme combinations are contemplated by this invention, which is not limited to the stated embodiments. For instance, specific enzymes may treat more than one stain and various enzyme combinations may act in synergy to further boost performance. A tablet may contain up to 5 to 6 different enzymes.

In the embodiments of the present invention the enzyme tablets are made from ingredients that eliminate the need to provide enzyme stabilizers. For instance, the enzyme tablets of the present invention do not contain bleaching agents, bleach activators, and the cleaning agent surfactants found in laundry and dish detergents. The tablets of the present invention need not contain enzyme stabilizers to protect against harsh substances found in laundry and dish detergents, such as bleaching agents, bleach activators and surfactants. Those skilled in the art will recognize that stabilized enzyme formulations may be used in the present invention but are not required thereby allowing the use of more economical enzyme formulations made with smaller amounts of, or no stabilizers and including lower amounts of, or no barrier materials and coating layers for enzymes in the form of granules.

In the embodiments of the present invention the enzyme tablets retain at least 80% stability, at least 90% stability, at least 95% stability, and at least 100% storage stability when stored at 45° C. and 80% relative humidity for at least three days and up to at least 80 days. The storage stability of the enzyme tablets of the present invention compared with enzyme tablets made with bleaching agents and bleach activators is at least 5% higher, at least 10% higher, at least 15% higher, at least 20% higher, at least 25% higher, at least 30% higher, at least 40% higher, at least 50% higher, at least 60% higher, at least 70% higher, at least 80% higher, at least 90% higher, and at least 100% higher when stored at 45° C. and 80% relative humidity for at least three days and up to at least 80 days.

In the embodiments of the present invention the enzyme tablets provide a cleaning benefit when compared to the cleaning benefit provided by commercially available laundry and dish detergent.

For purposes of the present disclosure, a “cleaning benefit” means that the wash and/or fabric care performance using the enzyme tablet of the present invention with a commercial laundry or dish detergent in an automatic washing machine under energy efficient operating conditions is better than or at least equal to the wash performance of the commercial laundry or dish detergent alone. The cleaning benefit may be an increased or equal wash performance or fabric care performance using a shorter wash cycle time, a lower wash cycle temperature, a shorter wash cycle time and a lower wash cycle temperature, or a conventional wash cycle. A cleaning benefit is achieved at shorter wash cycles and/or lower wash temperatures as compared to conventional wash cycles when the wash performance is at least equal to the wash performance of the control detergent used without the enzyme tablet in the shorter and/or lower temperature wash cycle. A cleaning benefit is achieved when using a regular, or normal wash temperature and a conventional wash cycle time the wash performance is improved.

“Energy efficient operating conditions” means a decrease in the time of a wash cycle (a shorter wash cycle), a decrease in the temperature of a wash cycle (lower temperature wash cycle), or a decrease in the time and the temperature of a wash cycle (shorter and lower temperature wash cycle) as compared to the convention, typical time and temperature settings of commercial automatic laundry and dish washing machines. Such cycles are for United States automatic washing machines typically about 12 minutes to about 18 minutes for a “Regular wash cycle setting at about 33° C. to about 43° C. (“Warm” setting). Such cycles are for European automatic washing machines typically about 100 minutes to about 140 minutes (“Normal” setting) at 40° C. to about 60° C. Such cycles are for automatic washing machines in Asia typically about 15 minutes at 25° C. A typical “Normal” European wash cycle for an automatic dish washing machine is conducted for about 62 minutes at a peak temperature of 50° C.

The wash cycles using the tablets of the present invention may be between about at least 50% shorter, at least about 60% shorter, at least about 70% shorter, at least about 80% shorter than the conventional European wash cycle. In one embodiment, the wash cycle is about 65% to about 80% shorter, or about 65 minutes to about 105 minutes shorter, than the typical European wash cycle time. The wash cycles may be conducted at temperatures that are about 30% lower, about 35% lower, about 40% lower than conventional wash cycle temperatures. In one embodiment, the temperature is about 33% lower, or about 20° C. lower, than typical wash cycle temperatures.

The cleaning benefit should be understood to mean that the wash performance achieved at the energy efficient operating conditions is at least substantially as good as the wash performance that would have resulted from use of the automatic washing machine at the typical time and temperature settings.

For purposes of the present disclosure, wash performance is measured by L*ab and Y*xy chromaticity values using a Minolta Colorimeter and is reported using delta reflectance (L*)=L* (post-wash)−L*(pre-wash), or delta reflectance (Y*)=Y*(post-wash)−Y*(pre-wash), or delta color (E*)=\sqrt{(ΔL*)²+(Δa*)²+(Δb*)²}. “Improved wash performance” is defined as a higher delta L*, a higher delta Y*, and/or a higher delta E* for post-wash measurements of washes relative to control washes without the enzyme tablet of the present invention. The present invention provides an improved wash performance at least about a 2% to about a 2500%, at least about a 2% to a 1500%, and at least about a 4% to about a 2500% higher Delta L*, Delta Y*, and/or Delta E* value(s) as compared to the Delta L*, Delta Y*, and/or Delta E* value(s) obtained without the addition of the enzyme tablet of the present invention. A substantially as good Delta L*, Delta Y*, and/or Delta E* value is about 0%.

Wash performance measurements are made on soiled fabric swatches made with standardized processes and stains purchased from qualified suppliers such as Scientific Services S/D Inc. (Sparrow Bush, N.Y.), Testfabrics, Inc. (West Pittston, Pa.) and the Center for Test Materials (Vlaardingen, Netherlands). Pre-wash measurements refer to L* and/or a* and or b*, or Y* and/or x* and/or y* measurements made on soiled swatches prior to being washed. Post-wash measurements refer to L* and/or a* and or b*, or Y* and/or x* and/or y* made on swatches after washing in the automatic washing machine.

For purposes of the present invention “fabric care performance” means pill removal, pill prevention, reduced pilling propensity, and/or color clarification of fabrics.

### Enzymes

Enzymes of the present invention include proteases, cellulases, lipases, phospholipases, cutinases, oxidases, oxygenases, transferases, reductases, hemicellulases, mannanases, amylases, esterases, isomerases, pectinases, lactases, peroxidases, pectate lyases, laccases and mixtures thereof. Preferred enzymes include those enzymes capable of hydrolyzing substrates (e.g., stains). These enzymes are known as hydrolases, which include, but are not limited to, proteases (bacterial, fungal, acid, neutral or alkaline), amylases (alpha or beta), lipases, cellulases, and mixtures thereof. Particularly preferred enzymes include those sold under the trade names Purafect, Purastar, Properase, Puradax, Clarase, Multifect, Maxacal, Maxapem, and Maxamyl by Genencor International (U.S. Pat. No. 4,760,025 and WO 91/06637); Alcalase, Savinase, Primase, Durazyme, Duramyl, Ovozyme, Polarzyme, and Termamyl sold by Novo Industries A/S (Denmark) Particularly preferred proteases are subtilisins. Cellulase is another preferred enzyme and particularly cellulases or cellulase components isolated from Trichoderma reesei, such as found in the product Clazinase and Puradax. Preferred amylases include alpha amylases obtained from Bacillus licheniformis.

The amount of the enzyme in the tablet may vary and generally provides a higher enzyme dose in wash cycles as compared to the enzyme dosage provided by a typical scoop of a commercial enzyme-containing detergent. By weight, the enzyme in one embodiment is about 0.15 to about 0.75% w/w of the tablet. The percentage of the enzyme plus any non-enzyme granule materials, or enzyme carrier materials, in the tablet may be up to 30%, up to 40%, up to 50%, up to 60%, up to 70%, up to 80%, and up to 90%, with pure, active enzyme percentages generally being between about 0.1% to about 15%. In one embodiment, the enzyme percentage is about 7.6%. The dosage of the pure enzyme in the tablet is selected to boost the cleaning power of the conventional cleaning product. For instance, the final concentration of pure or active enzyme may be selected to provide about 0.1 to about 50 ppm of active enzyme, about 0.11 to about 50 ppm, about 0.16 to about 50 ppm, about 2 to about 50 ppm, and about 5 to about 50 ppm per laundry or dish wash cycle. Those skilled in the art will recognize that enzyme concentrations above 50 ppm may be utilized in the present invention.

The enzyme component of the tablet may be provided as a granule if convenient and cost effective, and typically is a low cost granule without the more expensive ingredients needed to stabilize enzymes when stored in detergents. One such granule has enzyme coated on a salt seed and a relatively thin coating layer of salt. The enzyme component also may be dried powdered enzyme, or enzyme sprayed or plated onto a carrier such as sugar, starch or maltodextrin. The enzyme also may be added in a wet granulation process, as described in U.S. Pat. No. 6,852,336, which is incorporated in its entirety herein. The following non-limiting list of patent publications illustrate known enzyme formulations that may be utilized in the present invention and preferably are modified to reduce or eliminate enzyme stabilizers, including reduction or elimination of barrier materials and protective coatings: U.S. Pat. No. 4,689,297; U.S. Pat. No. 5,254,283; U.S. Pat. No. 5,324,649; EP656058B1; EP804532B1; U.S. Pat. No. 6,120,811; U.S. Pat. No. 6,248,706; U.S. Pat. No. 6,204,236; U.S. Pat. No. 6,423,517; U.S. Pat. No. 6,432,902; EP1124945B1; U.S. Pat. No. 6,413,749; U.S. Pat. No. 6,602,841; U.S. Pat. No. 6,534,466; EP1220887B1; U.S. Pat. No. 6,310,027; U.S. Pat. No. 7,018,821; U.S. Pat. No. 4,106,991; U.S. Pat. No. 6,924,133; U.S. Pat. No. 6,933,141; EP1324651B1; EP170360; EP458849; EP2787464; WO03/080827; WO97/29116; WO93/07260; WO02/0746; and WO96/38527.

### Tablets

The tablets of the invention may be any shape, such as round or spherical, elongated, ellipsoid, cube-shaped, or other geometrical shapes. Tablets are herein understood to mean any solid enzyme formulation that is easily handled by the consumer, which includes, but is not limited to capsules, pills, gel-tablets, and dissolvable papers or sheets of dissolvable material. The size of the tablet may vary as well, and typically should be selected for ease of handling by the consumer and, for safety reasons, to be generally at least slightly larger than pharmaceutical tablets. For example, in one embodiment the tablets are spherical balls having a diameter of about 18 mm. In general, diameters that make the tablets easy to handle are about 15 to about 36 mm. In another embodiment the tablets are rectangular with at least rounded ends. The rectangular embodiment is about 25 mm in length, about 9 mm wide, and about 5-20 mm thick. In general, ellipsoid and rectangular tablets are easily handled where the length is from about 15 to 50 mm, the width is about 5 to 30 mm, and the thickness is about 5 to 30 mm. Sheet and cloth tablet embodiments may be thin and larger in length and/or width. In use, the consumer removes the tablet from the packaging by hand and then drops it into the cleaning operation, for instance, into a washing machine, and the tablets should be sized to accommodate this expected use with ease.

The weight of the tablets may be from about 2 to 10 grams, from 2 to 15 grams, from about 2 to 20 grams, from about 2 to 25 grams, and from about 2 to 30 grams. The density of the tablets may be from about 0.8 to 1.7 g/cm3, from about 0.9 to about 1.1 g/cm3, and from about 1.3 to about 1.6 g/cm3.

The tablets can be pan, or spray coated with a water soluble, film-forming material, such as polyethylene glycol (PEG 500), to reduce dust, and improve appearance. Those skilled in the art will recognize that the tablets can be coated with time-release materials. Additionally, tablets with more than one enzyme may coat the different enzyme components with coatings selected to release different enzymes at different times during a wash cycle.

The tablets in the present invention are stable; however, packaging materials can be used to further extend the shelf life, improve shipping durability, or enhance consumer convenience. The tablets may be individually placed within a dissolvable packaging material, for example, polyvinyl alcohol film, that allows dropping of the tablet directly into the cleaning operation, such as a wash cycle of a washing machine or a dish washing machine. Tablets of the present invention were individually packaged in a heat sealed, polyester film package. The packaging material is selected to slow, or prevent adsorption of moisture. Any flexible barrier film, with a low moisture vapor transmission rate, such as polyethylene, polypropylene, polyvinyl chloride, nylon, or blends thereof, should be suitable.

The tablets may be covered in a shrink wrap process or they may be provided as blister packs, which are molded plastic sheets with backing material, for example a foil laminate, that allow the consumer to retrieve a tablet by pressing on it to release it from the packaging. The tablets may also be provided in sealed boxes, bottles or jars, any of which can be fitted with a dispenser feature. The outer packaging may include identification of the use of the tablet, for instance, for removing grass stains or for generally boosting the performance of a generic detergent.

The tablets may be packaged as a cleaning kit with color-coded tablets with different enzymes, the packaging material providing a key to identify each colored tablet by function.

### Other Tablet Ingredients

Because the enzyme tablets are not stored in cleaning products and there is no need to protect the enzyme from harsh detergent ingredients such as bleaching agents, many protective ingredients used in enzyme granules stored in detergents are not required. Such ingredients that are not needed include barrier materials, high levels of enzyme stabilizers, and bleach neutralizers. Of course some of these protective ingredients may be used in the tablets if desired and if cost effective.

The components of the enzyme tablets of the present invention are binders or fillers, lubricants, disintegrants, and optional ingredients such as effervescent systems, scents and color agents.

The binders and fillers may be starch, for instance, modified starches such as wheat starch, corn starch, potato starch, any of which can be used in native form, pregelatinized form, or partially pregelatinized form. Other binders/fillers that are suitable for the present invention include dextrin, maltodextrin, sugars (such as lactose, fructose, dextrose, glucose, sucrose, raffinose, trehalose, and maltose), and sugar alcohols such as sorbitol, mannitol and inositol. Other binders/fillers suitable for use in the tablets are cellulose, microcrystalline cellulose, and modified cellulose materials such as hydroxypropylmethyl cellulose (HPMC), methyl cellulose, hydroxybutylmethyl cellulose, sodium carboxymethyl cellulose, hydroxyethylmethyl cellulose, hydroxy ethyl cellulose, acrylic polymers, latexes and polyvinyl pyrrolidone. Phosphates and sulfates also may serve as binders or fillers, for example dibasic calcium phosphate, monocalcium phosphate, or calcium sulfate dihydrate. A combination of two or more binders/fillers can be used in the tablets of the present invention. Additional binders/fillers include carrageenan, gum arabic, guar gum, xanthan gum, locust bean gum, chitosan, gelatin, collagen, casein, polyaspartic acid and polyglutamic acid, all of which can be efficient binders at low levels to minimize expense. In embodiments of the present invention, economical binders/fillers such as lactose, dextrose, and dibasic calcium phosphate were utilized.

Lubricants and glidant agents that enhance tablet manufacture may be added to the tablet. As used herein “lubricants and glidants” mean any agent, which reduces surface friction, lubricates the surface of the tablet, decreases static electricity or reduces friability of the tablets. Lubricants and glidants also can serve as anti-agglomeration agents during tablet manufacture. Suitable lubricating agents include, but are not limited to, such agents as silica, silicon dioxide, talc, magnesium stearate, stearic acid, calcium stearate, sodium stearyl fumarate, and polyethylene glycol (PEG). In embodiments of the present invention, lubricants were magnesium stearate and polyethylene glycol.

Disintegrants are used to ensure that the tablet dissolves to release the enzyme to perform its cleaning and/or fabric care function. Any known disintegrant may be used, such as polyvinylpyrrolidone (PVP), polyvinylpolypyrrolidone (PVPP), carboxymethylcellulose, alginate, clays, native starch, and modified starch, such as sodium starch glycolate or crosslinked starch. A sodium starch glycolate, Primojel™ was used in tablets shown in the Examples.

Optional ingredients that provide consumer signals, such as effervescent systems and scents and perfumes, may be included in the tablets of the present invention. For example, effervescence may be provided when the tablet is added to liquid wash water by adding citric acid and sodium bicarbonate to the tablets, and those skilled in the art will recognize that other effervescent systems may be provided.

Other optional ingredients such as perfumes, scents and colorants may be added to the tablets. For instance, colorants may be dyes such as red lake #40 or blue lake #1, that are mixed in with the tablet ingredients prior to tabletting, generally at less than 0.01% (w/w). The tablets of the present invention may be color-coded for the convenience of the consumer, and for those embodiments that contain more than one type of enzyme tablet, the packaging material can include a key matching the color to the specific purpose of each tablet.

### Other Adjunct Ingredients

Adjunct ingredients may be added to the tablets of the present invention, including but not limited to: metallic salts, antioxidants, enzyme protecting agents/scavengers such as ammonium sulfate, ammonium citrate, urea, guanidine hydrochloride, guanidine carbonate, guanidine sulfonate, thiourea dioxide, monethyanolamine, diethanolamine, triethanolamine, amino acids such as glycine, sodium glutamate and the like, proteins such as bovine serum albumin, casein and the like.

### Processes for Making Enzyme Tablets

Tablets may be made by direct compression tabletting of mixtures of enzyme, fillers/binders, lubricants, and any other optional ingredients. The enzyme component is mixed thoroughly with the other tablet ingredients prior to entering the tablet machine. Ingredients are blended in any suitable mixing device, such as a twin shell blender or similar apparatus, or using any mixing method that results in blending of the tablet ingredients.

The mixtures are then compressed into tablets, using any tabletting device, such as a tablet press (Stokes Model R-4, Warminster, Pa.). Tablet presses generally have upper and lower shape-corresponding punches, which fit into a die from above and below the die. Mixed tablet material is filled into the die cavity and at least one of the punches, typically the upper punch, enters the die cavity. Pressure is applied to both the upper and lower punches. The action of the upper and lower punches moving toward each other, applies pressure to the material between the punches, thus forming a tablet.

A wide variety of tablet shapes can be made. Tablet shape is determined by the tooling of the punches. Compaction forces vary, depending on the punch geometry, type of instrument, and formulation used. Typical compaction forces can range from 0.2 kN to 22 kN.

Alternatively, tablets may be made using dry or wet granulation procedures as described in U.S. Pat. No. 6,852,336, which is hereby incorporated by reference herein in its entirety. The '336 patent states that dry granulation procedures may be utilized where one of the components has sufficient cohesive properties to be tabletted. The method mixes the ingredients with a lubricant, if required. The wet granulation procedure described mixes the dry ingredient using a twin shell blender or double-cone blender under shear mixing conditions and then adds solutions of a binding agent to the mixed powders to obtain a granulation.

Direct compression is preferred because it is a rapid, economical process that directly compresses the powdered materials of the tablet composition typically without modifying the physical nature of the components. A lubricant or glidant improves the rate of flow to prevent adhesion of the tablet material to the surface of the dies and punches of the tablet press. The process may include optional pre-compression where less than the full compression force is applied prior to final compression. This process allows for removal of entrapped air to prevent later relaxation of the tablets.

The tablet product of the present invention is defined to include capsules. Capsules can be of either hard or soft gel type and may be made using, for example, the following processes.

Hard capsules are made of a two part shell, composed primarily of gelatin, or cellulose derivatives, with optional plasticizers such as polyvinyl pyrrolidone and glycerin. The two parts that comprise a complete capsule are the “capsule body”, into which the active material is filled, and the “cap”, which fits snugly over the capsule body. Hard capsules are hard, and inflexible, as the name implies, typically containing about 15 percent moisture. The capsule body and cap are made in advance, or may be purchased from a capsule manufacturer. The active ingredient, such as formulated enzyme granules or enzyme powders with and without carrier materials, is mixed with a suitable diluent such as lactose, or starch, and then packed into the empty capsule body. The cap is then placed over the capsule body, to fully enclose the active ingredient. This process can be done manually, but is more commonly achieved by employing the use of a variety of commercially available capsule filling machines.

A second type of capsule is the soft gel capsule. A soft gel capsule is a one-piece, hermetically sealed soft shell, containing a liquid, a suspension or a semisolid, called fill. The soft gel shell is made of a film-forming material such as gelatin, and a water dispersible or water-soluble plasticizer, to impart flexibility. While soft capsules are very commonly made of gelatin, they can be made of other polymers, such as hydroxypropyl methylcellulose. Suitable liquid fill materials are vegetable oils, wax, or polyethylene glycols or other liquids that are compatible with the capsule composition. Soft gel capsules allow delivery of liquid enzyme, or delivery of dry enzyme particles, as a suspension. To manufacture soft gel capsules, the enzyme, or active ingredient is blended with the fill material. Simultaneously, the gelatin base is prepared, by blending the gelatin and plasticizing materials. Formation and filling of the capsule take place at the same time. The encapsulating machine forms the gelatin base into two thin films, which are fed between two rotating dies. The dies contain depressions in the shape and size of the capsules. A pump, which is synchronized with the dies, delivers the liquid fill material into the depressions, and the capsules are then sealed, by pressure, immediately after filling. Following encapsulation, soft gel capsules undergo a two-step drying process, accomplished in a tumble dryer or fluid bed dryer, followed by tray drying, or curing for several days.

### EXAMPLES

The present invention is illustrated by the examples listed below, which examples are for illustration only and are not meant to limit the scope of the invention. The attached Figures are meant to be considered as integral parts of the specification and description of the invention. All references cited are herein specifically incorporated by reference for all that is described therein. The following examples are offered to illustrate, but not to limit the claimed invention.

Those skilled in the art will recognize that other enzymes and enzyme combinations may be selected, that other tablet ingredients may be substituted or used, that the tablet may be made in other configurations and shapes, including sheet shapes, and that the tablets may be packaged in a variety of ways and kits.

The materials used in the wash study Examples below used the following commercially available soiled fabric swatches from Testfabrics, Inc. (West Pittson, Pa.); Scientific Services S/D Inc.(Sparrow Bush, N.Y.); and the Center for Test Materials (Vlaardingen, Netherlands).

| Swatch | | | |
|---|---|---|---|
| Name | Source | Stain | Material |
| EMPA 101 | Testfabrics | Carbon black/olive oil | Cotton |
| EMPA 104 | Testfabrics | Carbon black/olive oil | Cotton-polyester |
| EMPA 111 | Testfabrics | Blood | Cotton |
| EMPA 112 | Testfabrics | Cocoa | Cotton |
| EMPA 116 | Testfabrics | Blood/milk/ink | Cotton |
| EMPA 117 | Testfabrics | Blood/milk/ink | Cotton-polyester |
| EMPA 120 | Testfabrics | Grease/quartz/iron oxide | Cotton |
| EMPA 164 | Testfabrics | Grass | Cotton |
| EMPA 141-3 | Testfabrics | Lipstick (4.0 grams) | Cotton |
| EMPA 143-1 | Testfabrics | Makeup (1.0 gram) | Cotton |
| AS-10 | Testfabrics | Pigment/oil/milk | Cotton |
| PC-10 | Testfabrics | Pigment/oil/milk | Cotton |
| Wfk 20JE | Testfabrics | Tea with protein | Cotton-polyester |
| Wfk 20L | Testfabrics | Red wine, aged | Cotton-polyester |
| BGH | Scientific | Blood/grass/humus | Cotton-polyester |
| | Services | | |
| CIC | Scientific | Chocolate Ice Cream | Cotton-polyester |
| | Services | | |
| DS | Scientific | Dust-sebum | Cotton-polyester |
| | Services | | |
| Grass | Scientific | Grass | Cotton-polyester |
| | Services | | |
| CS-26 | Testfabrics | Corn starch, colored | Cotton |
| CS-28 | Testfabrics | Rice starch, colored | Cotton |
| Wfk 10J | Testfabrics | Coffee | Cotton |
| Wfk 10LI | Testfabrics | Red wine, aged | Cotton |
| CS-06 | Testfabrics | Salad dressing w/ | Cotton |
| | | natural black | |
| Wfk 10D | Testfabrics | Pigment/sebum | Cotton |
| Wfk 10J | Testfabrics | Tea | Cotton |
| Wfk 10N | Testfabrics | Egg yolk-pigment | Cotton |
| Wfk 10T | Testfabrics | Ketchup | Cotton |
| Wfk 10Z | Testfabrics | Chocolate | Cotton |
| Wfk 10SG | Testfabrics | Tomato beef sauce | Cotton |

Information about the detergents used in the wash studies in the examples is set out below:

| Brand | Manufacturer | Class | Enzymes | Bleach |
|---|---|---|---|---|
| All Liquid | Unilever | Inexpensive | No | No |
| Ariel | Procter & Gamble | Premium | Yes | Yes |
| Supercompact | | | | |
| Ariel Color | Procter & Gamble | Premium | Yes | No |
| Ariel | Procter & Gamble | Premium | Yes | Yes |
| Ariel Biological | Procter & Gamble | Premium | Yes | Yes |
| Calgonite | Reckitt Benckiser | | Yes | Yes |
| Cascade Tabs | Procter & Gamble | | Yes | Yes |
| Electrasol Tabs | Reckitt Benckiser | | Yes | Yes |
| General | Henkel | Premium | Yes | Yes |
| Dash | Procter & Gamble | Premium | Yes | Yes |
| Excel | Unilever | | | |
| IEC | | Inexpensive | No | No |
| IEC + bleach | | Inexpensive | No | Yes |
| Lanza Tres | Reckitt Benckiser | Inexpensive | | No |
| Nice | Nice Group | Inexpensive | No | |
| Purex Liquid | Dial Corporation | Inexpensive | No | No |
| Somat | Henkel | | Yes | Yes |
| Tandil Color | Dalli | Inexpensive | Yes | No |
| Tandil UltraPlus | Dalli | Inexpensive | Yes | Yes |
| Tide Liquid | Procter & Gamble | Premium | Yes | No |

### Example 1

### Enzyme Tablet Formulations

Enzyme tablets were prepared using the formulations below in Tables 1 through 6. Each tablet weighed 3 to 7 grams and was spherical in shape. The example formulations used Purafect or Properase protease enzyme and/or Purastar or OxAm amylase enzyme, both available from Genencor International, Inc. (Palo Alto, Calif.).

The enzyme component of the tablets described below was, for convenience, added as granules composed of 3% to 16% w/w active enzyme. Specifically, the formulations were made with 7.6% active enzyme and with 8.4% active enzyme.

These granules were made using a fluid bed process described in U.S. Pat. No. 5,324,649, which is hereby incorporated by reference herein in its entirety. The fluid bed process fluidized the core or seed material, sodium sulfate crystals, in a fluid bed coater. For the Example 1 formulations in Tables 1 through 5 below, the enzyme ultra filtrate concentrate, with 1% added PVA, was spray coated onto the seed. A final, thin outer coating of sodium sulfate and titanium dioxide comprised 10% w/w of the final granule and was spray coated over the enzyme layer. No barrier layers were used to make these granules and a relatively thin coating layer was applied to reduce dust. For the Example 1 formulation shown in Table 6, the enzyme ultra filtrate concentrates of protease and amylase, with sucrose and starch added, were spray coated onto the seed. Additional coatings, which comprise 30 to 40% (w/w) of the granule, and are composed of sucrose, starch, sodium sulfate, or cellulosic polymers, were then applied over the enzyme layer. As stated above, the enzyme component also may be a dried powder or the enzyme may be absorbed onto a carrier material.

TABLE 1

| Ingredient | % (w/w) |
|---|---|
| enzyme | 7.6 |
| microcrystalline cellulose (Avicel pH-200) | 29.2 |
| lactose (Hormel CFI 14158) | 62.2 |
| amorphous silica Silicron R G-602 | 0.3 |
| (Millennium Specialty Chem) | |
| magnesium stearate (Alfa Aesar A16244) | 0.6 |
| Total | 100.0 |

TABLE 2

| Ingredient | % (w/w) |
|---|---|
| enzyme | 7.6 |
| microcrystalline cellulose (Avicel pH-200) | 18.0 |
| dicalcium phosphate dihydrate, (Fisher | 73.9 |
| Scientific, C-135) | |
| magnesium stearate (Alfa Aesar A16244) | 0.5 |
| Total | 100.0 |

TABLE 3

| Ingredient | % (w/w) |
|---|---|
| enzyme | 7.6 |
| dicalcium phosphate dihydrate, (Fisher | 45.6 |
| Scientific, C-135) | |
| lactose (Hormel CFI 14158) | 45.6 |
| magnesium stearate (Alfa Aesar A16244) | 1.1 |
| Total | 100.0 |

TABLE 4

| Ingredient | % (w/w) |
|---|---|
| enzyme | 7.6 |
| dicalcium phosphate dihydrate, (Fisher | 44.8 |
| Scientific, C-135) | |
| lactose (Hormel CFI 14158) | 44.8 |
| partially gelatinized corn starch, (Starch | 2.5 |
| 1500, Colorcon) | |
| magnesium stearate (Alfa Aesar A16244) | 0.3 |
| Total | 100.0 |

TABLE 5

| Ingredient | % (w/w) |
|---|---|
| enzyme | 7.6 |
| dicalcium phosphate dihydrate (J T Baker, 1430-01) | 41.0 |
| lactose (Hormel CFI 14158) | 41.0 |
| magnesium stearate (Alfa Aesar A16244) | 1.0 |
| hydroxypropylcellulose (Klucel Type ECS, Aqualon) | 7.4 |
| sodium starch glycolate (Primojel, Generichem) | 1.8 |
| Total | 100.0 |

TABLE 6

| Ingredient | % (w/w) |
|---|---|
| enzyme 1 | 8.4 |
| enzyme 2 | 24.5 |
| dextrose (Cerelose, Corn Products) | 36.7 |
| microcrystalline cellulose (Vivapur 101, JRS Pharma) | 29.3 |
| magnesium stearate (Alfa Aesar A16244) | 1.1 |
| Total | 100.0 |

### Example 2

### European Wash Study Using a Decreased Wash Cycle Time

The tablets used for the European wash testing were made with the formula shown in Table 3 above, and included enzyme amounts to deliver 3 ppm Purafect protease, or 2 ppm OxAm amylase, to an 18 liter capacity wash cycle. 54 mg active protease was used, or 36 mg active amylase was used for each wash load.

European wash studies were conducted using a European style washer (Miele Novotronic horizontal axis automatic washer). The wash duration was 32 minutes and the wash temperature was 40° C. Agitation was 1000 rpm. The water hardness was 150-180 ppm as CaCO₃ (3:1, Ca/Mg ratio in R.O. water). 1.7 kg of ballast fabric, consisting of mixed fabrics (cotton, cotton-poly and polyester), was used.

Duplicate samples of the following commercially available soiled swatches were used to simulate a mixed soil load: EMPA's 101 carbon black/olive oil on cotton, 104 carbon black/olive oil cotton-poly, 111 blood on cotton, 112 cocoa on cotton, 116 blood/milk/ink on cotton and 117 blood/milk/ink on cotton-poly; AS-10 pigment/oil/milk on cotton; WFK 207E Tea with protein on cot-poly and WFK 20L Red Wine on cot-poly (Testfabrics Inc, Pittston, Pa.), Blood/Grass Humus, CS-26 colored corn starch, and CS-28 colored rice starch (Scientific Services S/D Inc, Sparrow Bush, N.Y.). The duplicates of each swatch type were included in each wash.

After air drying, the reflectance (L*) of each swatch was read in three places across the swatch, using a Minolta Colorimeter, set to measure L*ab chromaticity values. For each swatch, the delta reflectance is reported. (L*)=L*(post wash)−L*(pre wash)

Three detergents as follows were tested:

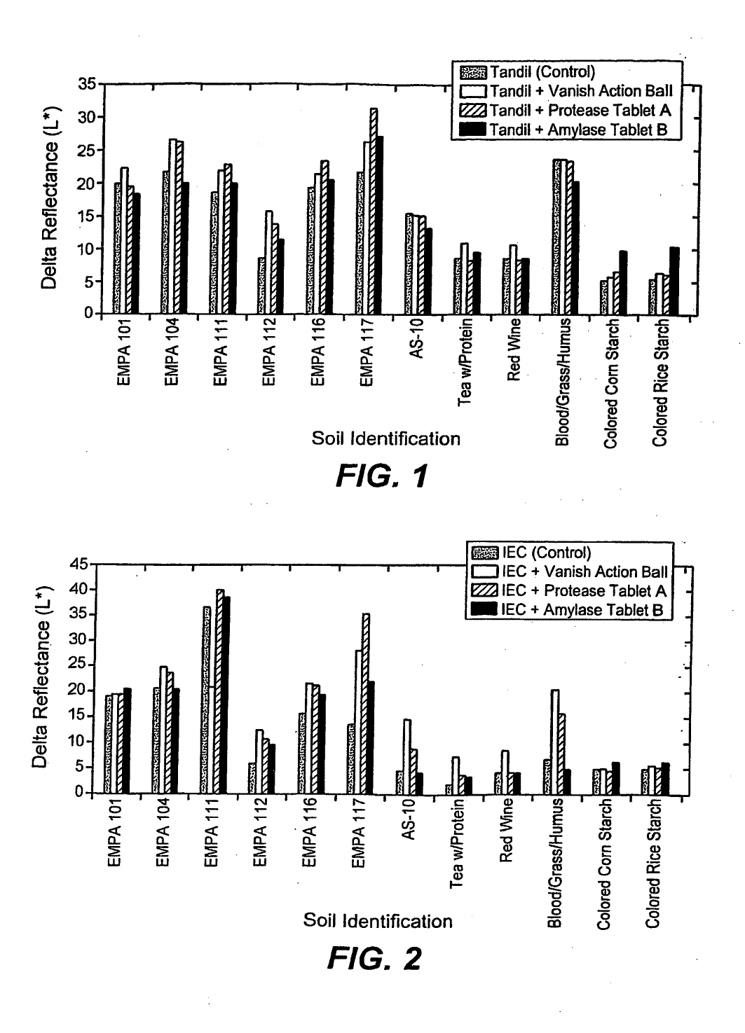

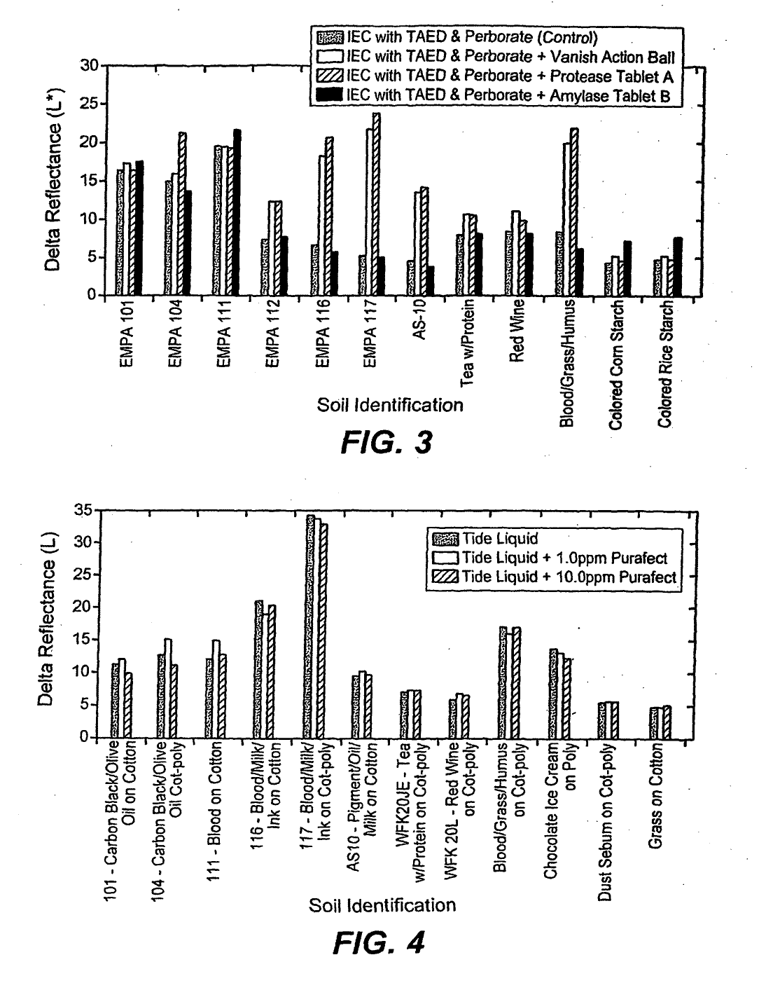

Tandil™ Ultra-Plus, Oxi Velkkenkracht™ (Dalli Benelux B.V.), dosed at 67.5 g/20 L.
IEC “unfinished (no TAED and or perborate), dosed at 100 g/20 L.
IEC “finished” (4% w/w TAED and 20% w/w perborate, dosed at 100 g/20 L.
For each of the three detergents tested, a wash test was done with no enzyme tablet (control), with a commercially available Vanish Action Ball™ laundry booster tablet containing bleaching components (Reckitt-Benckiser, Germany), with a protease-containing tablet of the present invention, and with an amylase-containing tablet of the present invention. The results are shown in graphs in FIGS. 1, 2 and 3 and in Tables 7, 8 and 9 below.

TABLE 7

| Performance (Delta L*) | | | | | Percent change in Delta L* | | |
|---|---|---|---|---|---|---|---|
| | | Tandil + | Tandil + | Tandil + | Tandil + | Tandil + | Tandil + |
| | Tandil | Vanish Action | Protease | Amylase | Vanish | Protease | Amylase |
| Stain | (control) | Ball | Tablet | Tablet | Action Ball | Tablet | Tablet |
| EMPA101 | 20.2 | 22.5 | 19.6 | 18.3 | 12 | −3 | −9 |
| EMPA104 | 21.8 | 26.8 | 26.5 | 20.2 | 23 | 22 | −7 |
| EMPA111 | 18.8 | 22.2 | 22.9 | 20.0 | 19 | 22 | 7 |
| EMPA112 | 8.9 | 15.7 | 13.9 | 11.6 | 77 | 57 | 31 |
| EMPA116 | 19.6 | 21.5 | 23.5 | 20.7 | 10 | 20 | 6 |
| EMPA117 | 21.8 | 26.3 | 31.6 | 27.3 | 21 | 45 | 25 |
| AS-10 | 15.4 | 15.4 | 15.2 | 13.3 | −1 | −1 | −14 |
| Tea/Protein | 8.9 | 11.1 | 8.5 | 9.5 | 25 | −4 | 8 |
| Red Wine | 8.6 | 10.7 | 8.6 | 8.6 | 24 | −1 | 0 |
| Blood/Grass/Humus | 23.9 | 23.8 | 23.4 | 20.5 | 0 | −2 | −14 |
| Chocolate Icecream | 22.8 | 25.7 | 23.8 | 23.3 | 13 | 4 | 2 |
| Colored Cornstarch | 5.5 | 25.7 | 6.7 | 9.8 | 371 | 22 | 80 |
| Colored Ricestarch | 5.61 | 6.4 | 6.2 | 10.5 | 14 | 11 | 87 |

TABLE 8

| Performance (Delta L*) | | | | | Percent change in Delta L* | | |
|---|---|---|---|---|---|---|---|
| | | IEC + | IEC + | IEC + | IEC + | IEC + | IEC + |
| | | Vanish | Protease | Amylase | Vanish | Protease | Amylase |
| Stain | IEC | Action Ball | Tablet | Tablet | Action Ball | Tablet | Tablet |
| EMPA101 | 19.1 | 19.3 | 19.3 | 20.5 | 1 | 1 | 7 |
| EMPA104 | 20.6 | 24.9 | 24.0 | 20.3 | 21 | 16 | −1 |
| EMPA111 | 37.0 | 21.0 | 40.3 | 38.8 | −43 | 9 | 5 |
| EMPA112 | 6.0 | 12.3 | 10.6 | 9.5 | 107 | 77 | 60 |
| EMPA116 | 15.8 | 21.4 | 21.4 | 19.5 | 36 | 35 | 23 |
| EMPA117 | 13.7 | 28.2 | 35.6 | 22.1 | 106 | 160 | 61 |
| AS-10 | 4.5 | 14.7 | 8.9 | 4.1 | 230 | 99 | −8 |
| Tea/Protein | 2.0 | 7.5 | 3.6 | 3.4 | 270 | 78 | 65 |
| Red Wine | 4.1 | 8.6 | 4.1 | 4.2 | 113 | 2 | 4 |
| Blood/Grass/Humus | 6.7 | 20.3 | 15.7 | 4.8 | 203 | 134 | −28 |
| Chocolate Icecream | 25.3 | 24.1 | 23.7 | 24.2 | −5 | −6 | −4 |
| Colored Cornstarch | 4.8 | 24.1 | 4.5 | 6.4 | 406 | −5 | 35 |
| Colored Ricestarch | 4.8 | 5.7 | 5.3 | 6.2 | 19 | 9 | 29 |

TABLE 9

| Performance (Delta L*) | | | | | Percent change in Delta L* | | |
|---|---|---|---|---|---|---|---|
| | | IEC (+ TAED | IEC (+ TAED | IEC (+ TAED | IEC (+ TAED | IEC (+ TAED | IEC (+ TAED |
| | IEC (+ TAED | and perborate) + | and perborate) + | and perborate + | and perborate) + | and perborate) + | and perborate) + |
| | and perborate) | Vanish Action | Protease | Amylase | Vanish Action | Protease | Amylase |
| Stain | (control) | Ball | Tablet | Tablet | Ball | Tablet | Tablet |
| EMPA101 | 16.7 | 17.5 | 16.6 | 17.9 | 5 | −1 | 7 |
| EMPA104 | 15.1 | 16.3 | 21.5 | 14.0 | 7 | 42 | −8 |
| EMPA111 | 19.8 | 19.8 | 19.4 | 22.1 | 0 | −2 | 11 |
| EMPA112 | 7.7 | 12.5 | 12.6 | 7.9 | 63 | 64 | 3 |
| EMPA116 | 6.9 | 18.5 | 21.0 | 6.0 | 169 | 205 | −13 |
| EMPA117 | 5.4 | 21.9 | 24.3 | 5.3 | 306 | 350 | −2 |
| AS-10 | 4.7 | 13.7 | 14.5 | 4.0 | 192 | 210 | −15 |
| Tea/Protein | 8.3 | 10.9 | 10.8 | 8.3 | 32 | 31 | 1 |
| Red Wine | 8.7 | 11.3 | 10.2 | 8.4 | 30 | 17 | −4 |
| Blood/Grass/Humus | 8.7 | 20.3 | 22.1 | 6.5 | 135 | 155 | −25 |
| Chocolate Icecream | 20.2 | 20.3 | 6.8 | 17.3 | 0 | −67 | −14 |
| Colored Cornstarch | 4.5 | 20.3 | 4.7 | 7.5 | 347 | 4 | 65 |
| Colored Ricestarch | 5.11 | 5.5 | 5.1 | 8.0 | 8 | 0 | 56 |

### Example 3

### North American Wash Studies Using Convention Wash Cycle Times and Temperatures

North American wash studies were conducted using a top loading, vertical axis automatic washer (Frigidaire Horizon 2000). The wash duration was 15 minutes and the wash temperature was 40° C. Agitation was normal/fast. The water hardness was 150 ppm as CaCO₃ (3:1, Ca/Mg ratio in R.O. water). 2.8 kg of ballast fabric, consisting of mixed fabrics (cotton, cotton-poly and polyester), was used.

Duplicate samples of the following commercially available soiled swatches were used to simulate a mixed soil load: EMPA's 101 carbon black/olive oil on cotton, 104 carbon black/olive oil cotton-poly, 111 blood on cotton, 116 blood/milk/ink on cotton, and 117 blood/milk/ink on cotton-poly; AS-10 pigment/oil/milk on cotton; WFK 20JE tea with protein on cot-poly and WFK 20L Red Wine on cot-poly (Testfabrics Inc, Pittston, Pa.), Blood/Grass Humus, chocolate ice cream, dust sebum, and grass (Scientific Services S/D Inc, Sparrow Bush, N.Y.). The duplicates of each swatch type were included in each wash. After air drying, the reflectance (L*) of each swatch was read in three places across the swatch, using a Minolta Colorimeter, set to measure L*ab chromaticity values. For each swatch, the delta reflectance is reported. (L*)=L*(post wash)−L*(pre wash).

Two following two detergents were tested:

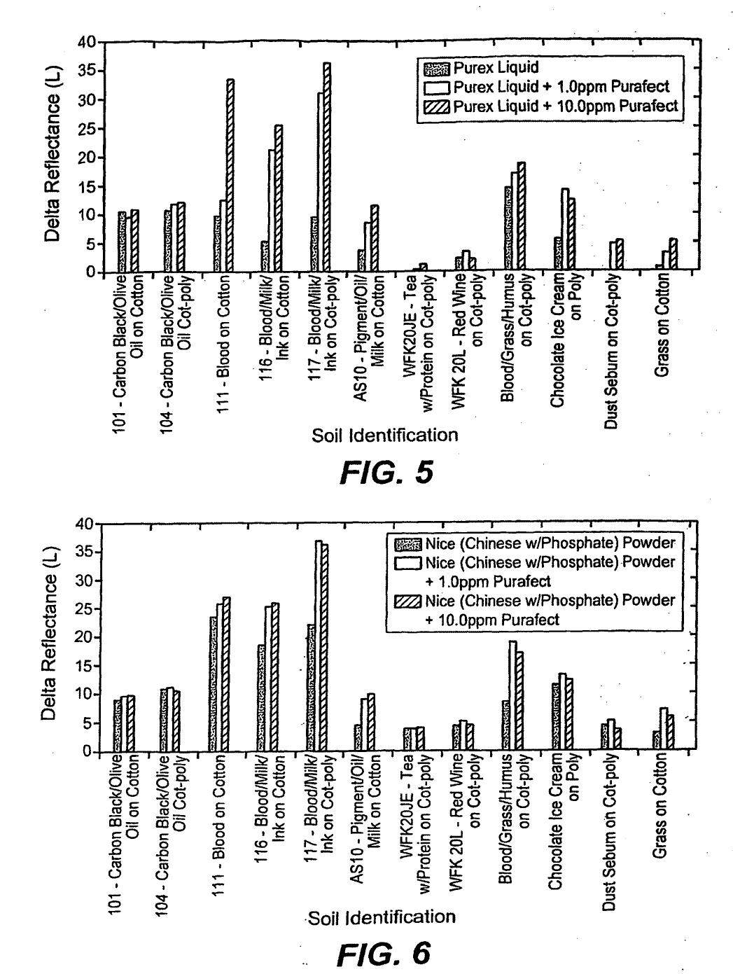

Tide® Liquid Detergent (Procter and Gamble, Cincinnati Ohio), dosed at 120 g/72 L.
Purex® Liquid Detergent (Dial Corp. Scottsdale, Ariz.), dosed at 150 g/72 L.
For each of the two detergents tested, a wash test was done with no added enzymes (control), with 1 ppm added Purafect protease, and with 10 ppm added Purafect protease.
The results are shown in graphs in FIGS. 4 and 5 and in Tables 10 and 11 below.

TABLE 10

| | Performance (Delta L*) | | | Percent change in Delta L* | |
|---|---|---|---|---|---|
| | Tide | Tide Detergent + | Tide Detergent + | Tide Detergent + | Tide Detergent + |
| Soil Type | Detergent | 1.0 ppm Protease | 10.0 ppm Protease | 1.0 ppm Protease | 10.0 ppm Protease |
| EMPA 101 | 11.2 | 12.0 | 9.8 | 7 | −13 |
| EMPA 104 | 12.7 | 15.1 | 11.1 | 19 | −13 |
| EMPA 111 | 12.0 | 14.9 | 12.8 | 24 | 7 |
| EMPA 116 | 20.9 | 18.9 | 20.3 | −10 | −3 |
| EMPA 117 | 34.2 | 33.7 | 32.8 | −1 | −4 |
| AS-10 | 9.5 | 10.3 | 9.7 | 8 | 2 |
| Wfk 20JE | 7.0 | 7.3 | 7.3 | 4 | 4 |
| Wfk 20L | 5.8 | 6.8 | 6.5 | 17 | 12 |
| Blood/grass/humus | 17.0 | 15.9 | 16.9 | −6 | −1 |
| Chocolate Ice Cream | 13.6 | 13.0 | 12.1 | −4 | −11 |
| Dust-sebum | 5.3 | 5.6 | 5.6 | 6 | 6 |
| Grass | 4.7 | 4.7 | 5.1 | 0 | 9 |

TABLE 11

| | Performance (Delta L*) | | | Percent change in Delta L* | |
|---|---|---|---|---|---|
| | Purex | Purex Detergent + | Purex Detergent + | Purex Detergent + | Purex Detergent + |
| Soil Type | Detergent | 1.0 ppm Protease | 10.0 ppm Protease | 1.0 ppm Protease | 10.0 ppm Protease |
| EMPA 101 | 10.4 | 9.5 | 10.8 | −9 | 4 |
| EMPA 104 | 10.5 | 11.7 | 11.9 | 11 | 13 |
| EMPA 111 | 9.6 | 12.5 | 33.5 | 30 | 249 |
| EMPA 116 | 5.2 | 21.1 | 25.4 | 306 | 388 |
| EMPA 117 | 9.3 | 31.0 | 36.2 | 233 | 289 |
| AS-10 | 3.6 | 8.4 | 11.5 | 133 | 219 |
| Wfk 20JE | −0.7 | 0.2 | 1.1 | −129 | −257 |
| Wfk 20L | 2.1 | 3.2 | 2.0 | 52 | −5 |
| Blood/grass/humus | 14.3 | 16.7 | 18.5 | 17 | 29 |
| Chocolate Ice Cream | 5.4 | 14.0 | 12.3 | 159 | 128 |
| Dust-sebum | −4.4 | 4.6 | 5.2 | −205 | −218 |
| Grass | 0.6 | 3.0 | 5.1 | 400 | 750 |

### Example 4

### Asian Wash Study Using Conventional Wash Time and Temperature

Asian wash studies were conducted using a top loading, vertical axis automatic washer (Frigidaire Horizon 2000). The wash duration was 15 minutes and the wash temperature was 25° C. Agitation was normal/fast. The water hardness was 150 ppm as CaCO₃ (3:1, Ca/Mg ratio in R.O. water). 2.8 kg of ballast fabric, consisting of mixed fabrics (cotton, cotton-poly and polyester), was used.

Duplicate samples of the following commercially available soiled swatches were used, to simulate a mixed soil load: EMPA's 101 carbon black/olive oil on cotton, 104 carbon black/olive oil cotton-poly, 111 blood on cotton, 116 blood/milk/ink on cotton, and 117 blood/milk/ink on cotton-poly; AS-10 pigment/oil/milk on cotton; WFK 20JE tea with protein on cot-poly and WFK 20L Red Wine on cot-poly (Testfabrics Inc, Pittston, Pa.), Blood/Grass Humus, chocolate ice cream, dust sebum, and grass (Scientific Services S/D Inc, Sparrow Bush, N.Y.). The duplicates of each swatch type were included in each wash. After air drying, the reflectance (L*) of each swatch was read in three places across the swatch, using a Minolta Colorimeter, set to measure L*ab chromaticity values. For each swatch, the delta reflectance is reported. (L*)=L*(post wash)−L*(pre wash)

Two following two detergents were tested:

Nice™ detergent powder, with phosphate, nil enzyme, (Nice Group, China), dosed at 96 g/72 L. Excel™ detergent powder (Unilever Thailand), dosed at 96 g/72 L.
For each of the two detergents tested, a wash test was done with no added enzymes (control), with 1 ppm added Purafect protease, and with 10 ppm added Purafect protease.

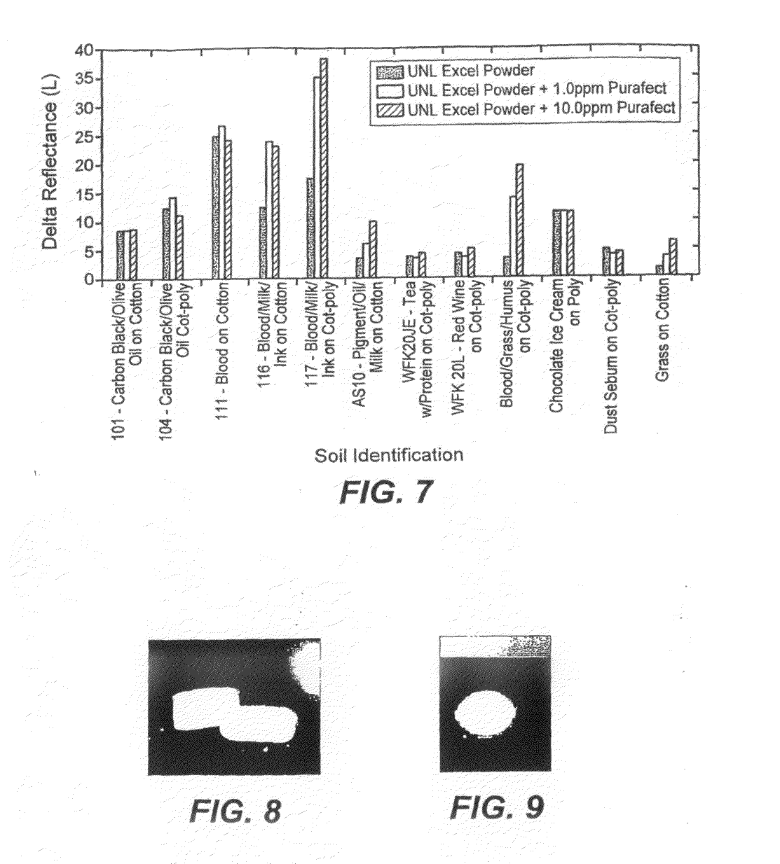

The results are shown graphically in FIGS. 6 and 7 and in Delta L* values used to create the graphs is presented below in tables 12 and 13.

TABLE 12

| | Performance (Delta L*) | | | Percent change in Delta L* | |
|---|---|---|---|---|---|
| | NICE | NICE Detergent + | NICE Detergent + | NICE Detergent + | NICE Detergent + |
| Soil Type | Detergent | 1.0 ppm Protease | 10.0 ppm Protease | 1.0 ppm Protease | 10.0 ppm Protease |
| EMPA 101 | 9.0 | 9.7 | 9.8 | 8 | 9 |
| EMPA 104 | 10.9 | 11.1 | 10.5 | 2 | −4 |
| EMPA 111 | 23.5 | 25.8 | 27.0 | 10 | 15 |
| EMPA 116 | 18.5 | 25.2 | 25.8 | 36 | 39 |
| EMPA 117 | 22.0 | 36.8 | 36.1 | 67 | 64 |
| AS-10 | 4.4 | 9.1 | 9.9 | 107 | 125 |
| Wfk 20JE | 3.8 | 3.7 | 4.0 | −3 | 5 |
| Wfk 20L | 4.3 | 5.2 | 4.4 | 21 | 2 |
| Blood/grass/humus | 8.5 | 18.9 | 17.0 | 122 | 100 |
| Chocolate Ice Cream | 11.4 | 13.2 | 12.2 | 16 | 7 |
| Dust-sebum | 4.3 | 5.2 | 3.7 | 21 | −14 |
| Grass | 3.1 | 7.1 | 5.9 | 129 | 90 |

TABLE 13

| | Performance (Delta L*) | | | Percent change in Delta L* | |
|---|---|---|---|---|---|
| | Excel | Excel Detergent + | Excel Detergent + | Excel Detergent + | Excel Detergent + |
| Soil Type | Detergent | 1.0 ppm Protease | 10.0 ppm Protease | 1.0 ppm Protease | 10.0 ppm Protease |
| EMPA 101 | 8.4 | 8.5 | 8.7 | 1 | 4 |
| EMPA 104 | 12.3 | 14.2 | 11.0 | 15 | −11 |
| EMPA 111 | 24.7 | 26.7 | 23.8 | 8 | −4 |
| EMPA 116 | 12.4 | 23.8 | 22.9 | 92 | 85 |
| EMPA 117 | 17.4 | 35.1 | 38.2 | 102 | 120 |
| AS-10 | 3.4 | 6.0 | 9.8 | 76 | 188 |
| Wfk 20JE | 3.6 | 3.3 | 4.1 | −8 | 14 |
| Wfk 20L | 4.1 | 3.4 | 5.0 | −17 | 22 |
| Blood/grass/humus | 3.3 | 13.8 | 19.4 | 318 | 488 |
| Chocolate Ice Cream | 11.3 | 11.3 | 11.2 | 0 | −1 |
| Dust-sebum | 4.7 | 3.8 | 4.3 | −19 | −9 |
| Grass | 1.4 | 3.5 | 6.1 | 150 | 336 |

The wash study results from Examples 2 through 4 demonstrate that the enzymes in high dosages, including the enzyme tablets of the present invention, improve wash performance when added to wash loads with generic detergents, such as Tandil™, IEC with and without bleach and enzymes, Purex® and Excel™. Of course one skilled in the art will recognize that providing enzyme in high dosages directly to the cleaning process, such as in the tablets of the present invention, may very well enhance high performance detergents where the existing enzyme content is insufficient to completely remove heavy stains.

The wash studies also show that the enzyme tablets of the present invention are useful in a variety of wash conditions and a variety of generic detergent brands. The enzyme tablets allowed for reduced energy consumption in Example 2 and provided an equal or improved wash performance compared to the detergent alone in the shorter wash cycle.

When compared to the Vanish™ balls, the protease tablet of the present invention was at least as effective as Vanish when tested on a variety of protease-sensitive stains (EMPA 111, 116, 117, AS-10, and BGH). The amylase tablet was superior to Vanish for amylase sensitive stains, such as corn starch and rice starch. The Vanish product showed the best results for removal of oxidizable stains such as teas and wine.

The wash studies shown in FIGS. 1-7 demonstrate that the wash performance using the enzyme tablets of the present invention is at least equal to or better than the wash results with the detergents alone.

### Example 5

### Dissolution or Disintegration Studies

Individual 5 g tablets were added to 1 liter of 25° C. de-ionized water, which was stirred using a 6 cm long stir bar and with a vortex of approximately 3 cm. Dissolution of the tablet was monitored visually, and the dissolution time was marked, at the point where the tablet had fully disintegrated, and larger pieces were no longer visible. The entire contents of the beaker were then immediately poured through a 1 mm screen, to verify that the tablet had fully disintegrated. The results are shown in Table 14 below.

TABLE 14

| | Dissolution Time |
|---|---|
| Formulation | (seconds) |
| Formulation 1 (see table 1) | 30 |
| Formulation 2 (see table 2) | 20 |
| Formulation 3 (see table 3) | 120 |
| Formulation 4 (see table 4) | 45 |
| Formulation 5 (see table 5) | 15 |

The dissolution studies show that the tablets of the present invention dissolve to release the enzyme.

### Example 6

### Tabletting Procedure

The dry ingredients of each of the tablets listed in Tables 1 through 6 may be blended in any suitable mixing device, such as a twin shell blender or similar apparatus, or in the case of the examples describe herein, small batches of 50 to 300 grams of dry ingredients were mixed by shaking them vigorously in a sealed plastic container.

The mixtures were then compressed into tablets, with a tablet press (Stokes Model R-4, Warminster, Pa.). The lower punch of the tablet press fits into a die from the bottom. Material is filled into the die cavity from above. After the material is filled, an upper punch, which has a corresponding shape, to the lower punch, enters the die cavity from above. Pressure is applied to both the upper and lower punches. The action of the upper and lower punches moving toward each other, applies pressure to the material between the punches, thus forming a tablet.

For the examples describe here, two punch geometries were used; a 0.95 cm×2.5 cm capsule shape, with beveled edges, and a 2 cm diameter spheroid shape. Compaction forces were believed to range from 0.2 kN to 22 kN.

### Example 7

### Enzyme Booster that Provides Grass Cleaning Benefit in Laundry

Enzyme tablets having the compositions given in Tables 1-6 are prepared to provide an enzyme booster which will provide 2-50 ppm of a single enzyme or a mixture of enzymes when added to a wash cycle. The tablets contain enzymes for cleaning grass stains with such enzymes being, for example, proteases, a mixture of proteases, plant cell wall degrading enzymes such as cellulases, hemicellulases, pectinases, and pectate lyases. Cleaning benefit results for grass stains are shown in FIGS. 1-6 for protease and amylase tablets. Additional grass stain cleaning benefit results are shown in Tables 7-13, 17, 21-22.

### Example 8

### Enzyme Booster That Provide Cleaning Benefits of Body Oil (Sebum) Stains in Laundry

Enzyme tablets having the compositions given in Tables 1-6 are prepared to provide an enzyme booster which will provide 2-50 ppm of a single enzyme or a mixture of enzymes when added to a wash cycle. The tablets contain enzymes for cleaning of body oil (sebum) stains which are sometimes referred to as dingy stains, with such enzymes being, for example, proteases or a mixture of proteases, lipases, phospholipases, and oxidases. Cleaning benefit results for sebum using protease and amylase tablets are shown in FIGS. 4 through 6 and in Tables 7-13, 17, 21-22.

### Example 9

### Enzyme Boosters that Provide Food Stain Cleaning Benefit in Laundry

Enzyme tablets having the compositions given in Tables 1-6 are prepared to provide an enzyme booster which will provide 0.1-50 ppm of a single enzyme or a mixture of enzymes when added to a wash cycle. The tablets will contain enzymes for cleaning food stains, with such enzymes being, for example, proteases, a mixture of proteases, amylases, a mixture of amylases, pectinases, pectate lyases, hemicellulases, mannanases, lipases, and oxidases. Food stain wash benefits using protease and amylase tablets are shown in FIGS. 1 through 6 and in Tables 7-13, 15-17, and 21-22.

### Example 10

### Enzyme Boosters that Provide Blood Stain Cleaning Benefit in Laundry

Enzyme tablets having the compositions given in Tables 1-6 are prepared to provide an enzyme booster which will provide 0.1-50 ppm of a single enzyme or a mixture of enzymes when added to a wash cycle. The tablets contain enzymes for cleaning blood stains, such as proteases, a mixture of proteases, lipases, and phospholipases. Cleaning benefits for removal of blood stains using protease and amylase enzyme tablets are shown in FIGS. 1 through 6 and in Tables 7-13, 17, 21-22.

### Example 11

### Enzyme Booster that Provide Cleaning Benefit in Automatic Dish Washing Compounds (ADW)

Enzyme tablets having the compositions given in Tables 1-6 are prepared to provide an enzyme booster which will provide 2-50 ppm of a single enzyme or a mixture of enzymes when added to a dish washer cleaning cycle. The tablets contain proteases, a mixture of proteases, amylases, a mixture of amylases, pectinases, pectate lyases, hemicellulases, mannanases, lipases, phospholipases, and oxidases.

The enzyme tablets used contained 1.12% w/w of protease enzyme and 0.6% w/w of amylase enzymes for a total dosage of 1.62% w/w enzyme. The dish washer used was a Miele G651 SC machine with wash cycle set to 50° Normal. This cycle runs at a peak temperature of 50° C. for 62 minutes. The wash performance measurements were made using egg yolk, egg yolk/milk, minced meat and rice milk stains on tableware as described in Industrieverband Korperpflege, and Waschmittel e.V (IKW), Larlstasse 21 D-60329 Frankfurt, Germany. The performance benefit was measured (visually or gravimetrically) on protease sensitive stains (egg yolk, egg yolk milk, minced meat) and amylase sensitive stains (rice milk). The results are shown below in Tables 15 and 16 with the mean value reported gravimetrically as a difference in weight.

TABLE 15
Wash Performance Results

| | | Egg Yolk Soil | | Egg Yolk/Milk |
|---|---|---|---|---|
| | | Removal | | Soil Removal |
| Samples | Mean | Benefit % | Mean | Benefit % |
| Calgonit 5in1 -AH | 45.6 | | 73.1 | |
| Calgonit 5in1 -AH + | 55.5 | 9.9 | 78.8 | 5.7 |
| enzyme tablet | | | | |
| Somat 5in1 -UK | 44.0 | | 72.7 | |
| Somat 5in1 -UK + | 55.6 | 11.6 | 75.6 | 2.9 |
| enzyme tablet | | | | |
| Cascade tabs 2006 | 41.2 | | 60.3 | |
| Cascade tabs 2006 + | 59.8 | 18.6 | 82.9 | 22.6 |
| enzyme tablet | | | | |
| Electrasol tabs 2006 | 32.9 | | 58.4 | |
| Electrasol tabs 2006 + | 60.6 | 27.7 | 82.8 | 24.4 |
| enzyme tablet | | | | |
| Enzyme tablet alone | 23.3 | | 39.3 | |

TABLE 16

| | | Minced Meat 140 | | Rice Milk 140 |
|---|---|---|---|---|
| | | Soil Removal | | Soil Removal |
| Samples | Mean | Benefit % | Mean | Benefit % |
| Calgonit 5in1 -AH | 7.3 | | 8.0 | |
| Calgonit 5in1 -AH + | 7.9 | 0.6 | 8.2 | 0.2 |
| enzyme tablet | | | | |
| Somat 5in1 -UK | 5.3 | | 7.2 | |
| Somat 5in1 -UK + | 7.9 | 2.6 | 9.0 | 1.8 |
| enzyme table | | | | |
| Cascade tabs 2006 | 4.9 | | 7.8 | |
| Cascade tabs 2006 + | 8.3 | 3.6 | 9.3 | 1.5 |
| enzyme tablet | | | | |
| Electrasol tabs 2006 | 4.1 | | 0.8 | |
| Electrasol tabs 2006 + | 8.3 | 4.2 | 6.4 | 5.6 |
| enzyme tablet | | | | |
| Enzyme tablet alone | 1.1 | | 5.3 | |

The wash study results demonstrate that the enzymes in high dosages, including the enzyme tablets of the present invention, improve wash performance when added to wash loads with generic automatic dish washing detergents, such as Calgonit™, SOMAT™, Cascade™

The wash study results also demonstrate that the enzymes in high dosages, including the enzyme tablets of the present invention, improve wash performance when added to two different types of commerciality available automatic dishwashing detergents that already contain protease and amylase.

### Example 12

### Enzyme Booster for any Non-Boron Containing Liquid Detergent Formulae

Enzyme tablets having the compositions given in Tables 1-6 are prepared to provide an enzyme booster which will provide 0.1-50 ppm of a single enzyme or a mixture of enzymes when added to a wash cycle. The tablets contain proteases, a mixture of proteases, amylases, a mixture of amylases, pectinases, pectate lyases, hemicellulases, mannanases, lipases, phospholipases, and oxidases. Cleaning benefits are shown in FIG. 6 and in Tables 11 and 18.

### Example 13

### Enzyme Booster that Provides Fabric Care Benefit in Laundry

Enzyme tablets having the compositions given in Tables 1-6 are prepared to provide an enzyme booster which will provide 0.1-50 ppm of a single enzyme or a mixture of enzymes when added to the wash. The tablets contain enzymes for fabric care, such as cellulases for cotton containing fabrics, cutinases or esterases (polyesterases) for polyester containing fabrics, and proteases for wool or silk containing fabrics. Fabric care benefits using enzymes are known in the art and are shown, for example, in U.S. Pat. No. 6,254,645 and in WO99/01604, both of which are hereby incorporated by reference in their entirety for cutinases and esterases (polyesterase). These publications show that cutinases, esterases (polyesterase), and lipases provide fabric care benefits such as reduction in pilling, color clarification, and modification of the surface properties of fabric.

Fabric care benefits using cellulases are known in the art and are shown, for example, in the following U.S. Pat. Nos. 6,921,655; 6,620,605; 6,767,879; 6,187,740; and 5,520,838 describing benefits such as antiredeposition of dyes, pilling prevention, and color clarification.

### Example 14

### North America Reduced Temperature Wash Study

Reduced Temperature wash studies were conducted using a top loading, vertical axis automatic washer (Kenmore; Ultra Clean Super 14). The wash duration was 15 minutes. “Cold” and “warm” wash temperatures were 24° C. and 38° C. respectively. Agitation was set to Heavy Duty Fast/Fast. The water hardness was 150 ppm as CaCO₃ (3:1, Ca/Mg ratio in reverse osmosis water). 2.5 kg of ballast fabric, consisting of mixed fabrics (cotton, cotton-poly and polyester), was used. Duplicate swatches were chosen (see Table) to simulate a mixed soil load. Post wash measurements were made on swatches allowed to air dry for at least four hours.

Two following two detergents were tested:

Purex® Liquid Detergent (Dial Corp. Scottsdale, Ariz.), dosed at 150 g/72 L.
All® Liquid Detergent (Unilever, Trumbull, Conn.) dosed at 131/72 L.
For each of the two detergents tested, a wash test was done with no added enzymes at 37.8° C.—“Warm” (control) and with 1 or 10 ppm added Purafect protease, or 1 and 10 ppm PuraStar OxAm amylase at 23.9° C.—“Cold” temperatures. Enzymes were added as liquid samples.

The results are shown in Tables 17-20.

TABLE 17

| | Performance (Delta E*) | | | | Percent change in Delta E* | | |
|---|---|---|---|---|---|---|---|
| | All Detergent - | All Detergent - | All Detergent + | All Detergent + | All Detergent - | All Detergent + | All Detergent + |
| | warm wash | cold wash | 1.0 ppm Amylase - | 10.0 ppm Amylase - | cold wash | 1.0 ppm Amylase - | 10.0 ppm Amylase - |
| Soil Type | control | control | cold wash | cold wash | control | cold wash | cold wash |
| PC-10 | 6.1 | 4.4 | 5.6 | 8.7 | −28 | −8 | 43 |
| EMPA 101 | 16.5 | 7.3 | 8.0 | 8.0 | −56 | −52 | −52 |
| EMPA 104 | 13.7 | 6.6 | 7.0 | 6.4 | −52 | −49 | −53 |
| EMPA 111 | 18.1 | 19.6 | 20.2 | 20.5 | 8 | 12 | 13 |
| EMPA 112 | 6.9 | 3.7 | 4.6 | 8.0 | −46 | −33 | 16 |
| EMPA 116 | 10.0 | 6.2 | 8.5 | 12.2 | −38 | −15 | 22 |
| EMPA 117 | 7.3 | 3.8 | 6.5 | 10.4 | −48 | −11 | 42 |
| Wfk 10J | 8.9 | 9.3 | 9.7 | 9.4 | 4 | 9 | 6 |
| Wfk 10LI | 21.0 | 19.9 | 19.5 | 20.2 | −5 | −7 | −4 |
| Grass | 19.9 | 13.3 | 16.7 | 18.7 | −33 | −16 | −6 |
| Blood/grass/ | 16.3 | 11.1 | 13.2 | 21.8 | −32 | −19 | 34 |
| humus | | | | | | | |
| Chocolate | 13.6 | 8.4 | 12.4 | 16.0 | −38 | −9 | 18 |
| Ice Cream | | | | | | | |
| Dust-sebum | 16.1 | 11.5 | 15.3 | 13.8 | −29 | −5 | −14 |
| CS-26 | 17.2 | 13.4 | 23.2 | 31.9 | −22 | 35 | 85 |
| CS-28 | 17.4 | 13.8 | 22.3 | 30.9 | −21 | 28 | 78 |

TABLE 18

| | Performance (Delta E*) | | | | | Percent change in Delta E* | |
|---|---|---|---|---|---|---|---|
| | All Detergent - | All Detergent - | All Detergent + | All Detergent + | All Detergent - | All Detergent + | All Detergent + |
| | warm wash | cold wash | 1.0 ppm Protease - | 10.0 ppm Protease - | cold wash | 1.0 ppm Protease - | 10.0 ppm Protease - |
| Soil Type | control | control | cold wash | cold wash | control | cold wash | cold wash |
| PC-10 | 6.1 | 4.4 | 14.0 | 12.3 | −28 | 130 | 102 |
| EMPA 101 | 16.5 | 7.3 | 7.4 | 7.2 | −56 | −55 | −56 |
| EMPA 104 | 13.7 | 6.6 | 6.9 | 7.3 | −52 | −50 | −47 |
| EMPA 111 | 18.1 | 19.6 | 21.9 | 26.9 | 8 | 21 | 49 |
| EMPA 112 | 6.9 | 3.7 | 9.4 | 11.6 | −46 | 36 | 68 |
| EMPA 116 | 10.0 | 6.2 | 15.5 | 20.5 | −38 | 55 | 105 |
| EMPA 117 | 7.3 | 3.8 | 24.1 | 31.1 | −48 | 230 | 326 |
| Wfk 10J | 8.9 | 9.3 | 9.6 | 10.1 | 4 | 8 | 13 |
| Wfk 10LI | 21.0 | 19.9 | 20.4 | 19.8 | −5 | −3 | −6 |
| Grass | 19.9 | 13.3 | 25.1 | 25.2 | −33 | 26 | 27 |
| Blood/grass/ | 16.3 | 11.1 | 29.2 | 28.4 | −32 | 79 | 74 |
| humus | | | | | | | |
| Chocolate | 13.6 | 8.4 | 22.5 | 24.2 | −38 | 65 | 78 |
| Ice Cream | | | | | | | |
| Dust-sebum | 16.1 | 11.5 | 13.2 | 11.3 | −29 | −18 | −30 |
| CS-26 | 17.2 | 13.4 | 15.4 | 14.3 | −22 | −10 | −17 |
| CS-28 | 17.4 | 13.8 | 14.6 | 14.9 | −21 | −16 | −14 |

TABLE 19

| | Performance (Delta E*) | | | | Percent change in Delta E* | | |
|---|---|---|---|---|---|---|---|
| | Purex | Purex | Purex | Purex | Purex | Purex | Purex |
| | Detergent - | Detergent - | Detergent + | Detergent + | Detergent - | Detergent + | Detergent + |
| | warm wash | cold wash | 1.0 ppm Amylase - | 10.0 ppm Amylase - | cold wash | 1.0 ppm Amylase - | 10.0 ppm Amylase - |
| Soil Type | control | control | cold wash | cold wash | control | cold wash | cold wash |
| PC-10 | 6.1 | 4.4 | 4.5 | 8.3 | −28 | −26 | 36 |
| EMPA101 | 12.2 | 7.1 | 6.8 | 7.2 | −42 | −44 | −41 |
| EMPA104 | 14.0 | 5.6 | 5.9 | 7.0 | −60 | −58 | −50 |
| EMPA111 | 17.3 | 18.3 | 18.6 | 19.5 | 6 | 8 | 13 |
| EMPA112 | 6.5 | 3.5 | 4.6 | 5.2 | −46 | −29 | −20 |
| EMPA116 | 5.7 | 4.5 | 6.4 | 9.8 | −21 | 12 | 72 |
| EMPA117 | 7.0 | 3.8 | 4.2 | 8.0 | −46 | −40 | 14 |
| Wfk 10J | 9.3 | 9.4 | 9.8 | 9.8 | 1 | 5 | 5 |
| Wfk10L1 | 20.1 | 19.3 | 18.3 | 18.5 | −4 | −9 | −8 |
| Grass | 13.6 | 10.0 | 11.0 | 12.1 | −26 | −19 | −11 |
| Blood/Grass/ | 14.1 | 12.0 | 11.7 | 14.8 | −15 | −17 | 5 |
| Humus | | | | | | | |
| Chocolate | 11.4 | 9.1 | 9.4 | 10.2 | −20 | −18 | −11 |
| Icecream | | | | | | | |
| Dust/sebum | 11.9 | 9.6 | 9.9 | 9.8 | −19 | −17 | −18 |
| CS-26 | 16.7 | 14.9 | 23.9 | 30.7 | −11 | 43 | 84 |
| CS-28 | 17.9 | 13.8 | 21.9 | 31.3 | −23 | 22 | 75 |

TABLE 20

| | Performance i(Delta E*) | | | | Percent change in Delta E* | | |
|---|---|---|---|---|---|---|---|
| | Purex | Purex | Purex | Purex | Purex | Purex | Purex |
| | Detergent - | Detergent - | Detergent + | Detergent + | Detergent - | Detergent + | Detergent + |
| | warm wash | cold wash | 1.0 ppm Protease - | 10.0 ppm Protease - | cold wash | 1.0 ppm Protease - | 10.0 ppm Protease - |
| Soil type | control | control | cold wash | cold wash | control | cold wash | cold wash |
| PC-10 | 6.1 | 4.4 | 16.6 | 16.6 | −28 | 172 | 172 |
| EMPA101 | 12.2 | 7.1 | 8.3 | 6.8 | −42 | −32 | −44 |
| EMPA104 | 14.0 | 5.6 | 6.0 | 5.8 | −60 | −57 | −59 |
| EMPA111 | 17.3 | 18.3 | 22.4 | 26.2 | 6 | 29 | 51 |
| EMPA112 | 6.5 | 3.5 | 7.9 | 9.7 | −46 | 22 | 49 |
| EMPA116 | 5.7 | 4.5 | 16.5 | 21.7 | −21 | 189 | 281 |
| EMPA117 | 7.0 | 3.8 | 23.7 | 29.7 | −46 | 239 | 324 |
| Wfk 10J | 9.3 | 9.4 | 9.3 | 10.0 | 1 | 0 | 8 |
| Wfk 10L1 | 20.1 | 19.3 | 19.2 | 18.8 | −4 | −4 | −6 |
| Grass | 13.6 | 10.0 | 19.4 | 22.1 | −26 | 43 | 63 |
| Blood/grass/ | 14.1 | 12.0 | 27.2 | 30.7 | −15 | 93 | 118 |
| humus | | | | | | | |
| Chocolate | 11.4 | 9.1 | 24.5 | 23.3 | −20 | 115 | 104 |
| Icecream | | | | | | | |
| Dust/sebum | 11.9 | 9.6 | 10.2 | 10.3 | −19 | −14 | −13 |
| CS-26 | 16.7 | 14.9 | 14.7 | 14.2 | −11 | −12 | −15 |
| CS-28 | 17.9 | 13.8 | 14.9 | 14.6 | −23 | −17 | −18 |

### Example 15

### European Wash Study Using Conventional Wash Times and Temperatures

The tablets used for the European wash testing were made with the formula shown in Table 3 above, and included enzyme amounts to deliver 3 ppm Purafect protease and 2 ppm OxAm amylase, to an 18 liter capacity wash cycle. 54 mg active protease was used, or 36 mg active amylase was used for each wash load.

European wash studies were conducted using a European style washer (Miele Novotronic horizontal axis automatic washer, model W1918). The wash duration was 114 minutes and the wash temperature was 40° C. Agitation was 1000 rpm. The water hardness was 250 ppm as CaCO₃ (3:1, Ca/Mg ratio in R.O. water). 2.0 kg of ballast fabric, consisting of mixed fabrics (cotton, cotton-poly and polyester), was used. Duplicate swatches were chosen (see Table) to simulate a mixed soil load. Post wash measurements were made on swatches allowed to air dry for at least four hours.

The detergents used tested was

Lanza Tres™ HDD (Reckitt-Benckiser, Itala S.p.A.), dosed at 137 g/18 L
A wash test was done with no enzyme tablet (control) and with a protease and amylase-containing tablet of the present invention.

The results are shown in Table 21.

TABLE 21

| | Performance (Delta L*) | | |
|---|---|---|---|
| | | Lanza Detergent + Enzyme | Percent |
| | Lanza | tablet (3.0 ppm protease/ | change in |
| Soil Type | Detergent | 2.0 ppm amylase) | Delta L* |
| PC-10 | 28.6 | 29.0 | 1.4 |
| Grass | 18.7 | 27.2 | 13.4 |
| EMPA 101 | 18.6 | 19.4 | 4.3 |
| EMPA 104 | 18.6 | 18.6 | 0.0 |
| EMPA 111 | 50.6 | 55.9 | 10.5 |
| EMPA 112 | 8.7 | 14.7 | 69.0 |
| EMPA 116 | 15.9 | 23.8 | 49.7 |
| EMPA 117 | 16.5 | 39.6 | 140.0 |
| EMPA 120 | 30.1 | 31.6 | 5.0 |
| EMPA 141-3 | 10.1 | 9.7 | −4.0 |
| EMPA 143-1 | 10.7 | 11.1 | 3.7 |
| Dust-sebum | 32.4 | 33.3 | 2.8 |
| Blood/grass/humus | 57.0 | 55.9 | −1.9 |
| Coffee | 16.1 | 16.4 | 1.9 |
| Chocolate Ice | 28.3 | 30.7 | 8.5 |
| Cream | | | |
| Wfk 10J | 14.0 | 14.3 | 2.1 |
| CS-26 | 6.5 | 12.9 | 98.5 |

### Example 16

### European Reduced Wash Time Study

European wash studies were conducted using a European style washer (Miele Novotronic horizontal axis automatic washer). Wash temperature was 40° C. Standard wash duration was 114 minutes, indicated by “Normal” by the washing machine programming, and reduced cycle was 32 minutes, designated as “Rapid” by the washing machine. Agitation was 1000 rpm. Water Hardness was supplemented to approximately 250 ppm as CaCO₃ (3:1, Ca/Mg ratio in R.O. water). 2.0 kg of ballast fabric, consisting of mixed fabrics (cotton, cotton-poly and polyester), was used. Duplicate swatches were chosen (see Table) to simulate a mixed soil load. Post wash measurements were made on swatches allowed to air dry for at least four hours.

Two detergents were tested:

Lanza Tres™ HDD (Reckitt-Benckiser, Itala S.p.A.), dosed at 172 g/18 L
Ariel Biological HDD (Procter and Gamble, Cincinatti Ohio), dosed at 95 g/18 L.

For each of the detergent/temperatures tested a wash test was done with no tablet for “normal” and “rapid” wash cycle durations, and with a protease/amylase tablet for “rapid” wash cycle duration.

The results are shown in Tables 22 and 23.

TABLE 22

| | Performance (Delta E*) | | | Percent change in Delta E* | |
|---|---|---|---|---|---|
| | | | Lanza Detergent | | Lanza Detergent |
| | Lanza Detergent - | Lanza Detergent - | 32 minutes + | Lanza Detergent - | 32 minutes + |
| Soil Type | 154 minutes | 32 minutes | Enzyme Tablet | 32 minutes | Enzyme Tablet |
| EMPA 101 | 27.1 | 20.1 | 21.5 | −26 | −21 |
| EMPA 104 | 31.8 | 21.7 | 22.1 | −32 | −31 |
| EMPA 111 | 44.2 | 37.5 | 42.3 | −15 | −4 |
| EMPA 112 | 13.4 | 7.6 | 11.6 | −43 | −13 |
| EMPA 116 | 26.6 | 14.3 | 22.0 | −46 | −17 |
| EMPA 117 | 37.3 | 15.3 | 36.0 | −59 | −3 |
| EMPA 120 | 38.6 | 26.3 | 29.6 | −32 | −23 |
| EMPA 141-3 | 21.2 | 12.4 | 12.0 | −42 | −43 |
| EMPA 143-1 | 12.4 | 8.8 | 9.5 | −29 | −23 |
| Coffee | 18.8 | 14.7 | 14.7 | −22 | −22 |
| EMPA 164 | 12.8 | 5.9 | 8.7 | −54 | −32 |
| Wfk 10N | 2.7 | 1.0 | 4.3 | −63 | 59 |
| Wfk 10T | 13.1 | 9.6 | 10.3 | −27 | −21 |
| Wfk 10Z | 7.1 | 3.1 | 3.3 | −56 | −54 |
| CS-06 | 7.0 | 4.1 | 5.9 | −41 | −16 |
| CS-26 | 19.5 | 15.5 | 28.1 | −21 | 44 |
| Wfk 10D | 11.8 | 7.7 | 10.5 | −35 | −11 |
| Wfk 10J | 19.1 | 8.6 | 8.8 | −55 | −54 |
| Wfk 10LI | 22.8 | 17.8 | 17.8 | −22 | −22 |
| Wfk 10SG | 16.6 | 14.4 | 12.7 | −13 | −23 |

TABLE 23

| | Performance (Delta E*) | | | Percent change in Delta E* | |
|---|---|---|---|---|---|
| | | | Ariel Biological | | Ariel Biological |
| | Ariel Biological | Ariel Biological | Detergent | Ariel Biological | Detergent |
| | Detergent - | Detergent - | 32 minutes + | Detergent - | 32 minutes + |
| Soil Type | 154 minutes | 32 minutes | Enzyme Tablet | 32 minutes | Enzyme Tablet |
| EMPA 104 | 26.5 | 15.2 | 16.0 | −43 | −40 |
| EMPA 111 | 41.0 | 30.0 | 30.2 | −27 | −26 |
| EMPA 112 | 17.2 | 10.5 | 13.1 | −39 | −24 |
| EMPA 116 | 18.9 | 13.1 | 20.1 | −31 | 6 |
| EMPA 117 | 30.3 | 15.7 | 29.9 | −48 | −1 |
| EMPA 120 | 35.8 | 24.1 | 22.3 | −33 | −38 |
| EMPA 141-3 | 20.9 | 12.2 | 10.7 | −42 | −49 |
| EMPA 143-1 | 12.4 | 8.6 | 7.4 | −31 | −40 |
| EMPA 164 | 13.3 | 5.1 | 9.0 | −62 | −32 |
| Wfk 10N | 8.8 | 4.5 | 12.7 | −49 | 44 |
| Wfk 10T | 13.4 | 10.3 | 10.5 | −23 | −22 |
| Wfk 10Z | 8.0 | 1.8 | 4.3 | −78 | −46 |
| CS-06 | 12.3 | 7.8 | 8.9 | −37 | −28 |
| CS-26 | 27.5 | 18.2 | 26.8 | −34 | −3 |
| Wfk 10D | 13.0 | 10.0 | 8.6 | −23 | −34 |
| Wfk 10J | 20.2 | 11.7 | 11.3 | −42 | −44 |
| Wfk 10LI | 23.9 | 18.9 | 23.1 | −21 | −3 |
| Wfk 10SG | 23.3 | 14.6 | 11.1 | −37 | −52 |

### Example 17

### European Wash Study Using Conventional Wash Times and Temperatures with Capsules

The capsule form of the tablets of the present invention used for the European wash testing were made according to Table 24 formulation below, and included enzyme amounts to deliver 3 ppm Purafect protease and 2 ppm OxAm amylase, to an 18 liter capacity wash cycle. Dry capsule ingredients were blended and then manually filled into hard gelatin capsules, size 000 (Capsuline, Inc., Pompano Beach, Fla.) 54 mg active protease was used, or 36 mg active amylase was used for each wash load.

European wash studies were conducted exactly like Example 15 using a European style washer (Miele Novotronic horizontal axis automatic washer, model W1918). The wash duration was 114 minutes and the wash temperature was 40° C. Agitation was 1000 rpm. The water hardness was 250 ppm as CaCO₃ (3:1, Ca/Mg ratio in R.O. water). 2.0 kg of ballast fabric, consisting of mixed fabrics (cotton, cotton-poly and polyester), was used. Duplicate swatches were chosen to simulate a mixed soil load. Post wash measurements were made on swatches allowed to air dry for at least four hours.

The detergent used was 109 g IEC+6 g TAED+29 g perborate,

A wash test was done with no enzyme capsule (control) and with a protease and amylase-containing capsule of the present invention.

The results are shown in Table 25. This experiment demonstrates that an alternate capsule format can be used.

TABLE 24

| Ingredient | % (w/w) |
|---|---|
| enzyme 1 | 15.3 |
| enzyme 2 | 44.7 |
| dextrose (Cerelose, Corn Products) | 40 |
| Total | 100.0 |

TABLE 25

Performance (Delta E*)

| | | capsule; 3 ppm | Percent |
|---|---|---|---|
| | wash | protease, 3 ppm | increase in |
| Soil Type | control | amylase | Delta E |
| Empa 104 | 31.4 | 28.4 | −10 |
| Empa 111 | 37.5 | 44.0 | 17 |
| Empa 112 | 13.1 | 21.6 | 64 |
| Empa 116 | 3.1 | 25.0 | 705 |
| Empa 117 | 3.9 | 41.2 | 959 |
| Empa 120 | 36.3 | 31.5 | −13 |
| Empa 141-3 | 22.3 | 20.9 | −6 |
| Empa 143-1 | 10.4 | 10.6 | 2 |
| Empa 164 | 7.2 | 17.1 | 136 |
| 10T | 11.2 | 12.6 | 12 |
| 10Z | 7.1 | 10.1 | 43 |
| CS-06 | 5.3 | 7.2 | 37 |
| CS-26 | 14.9 | 34.3 | 130 |
| 10D | 10.7 | 12.1 | 14 |
| 10J | 19.2 | 19.7 | 3 |
| 10LI | 30.2 | 30.3 | 0 |
| 10SG | 17.0 | 19.5 | 15 |
| Butter | 29.0 | 27.0 | −7 |

### Example 18

### Stability Studies

The stability of enzyme tablets of the present invention was tested using a protease enzyme in the form or granules added to the other tablet ingredients and compressed into tablets. The formulation of the tablets is listed below in Table 26.

TABLE 26
Composition of Enzyme Tablets C

| Ingredient | % (w/w) |
|---|---|
| Protease granules | 7.6 |
| calcium phosphate dibasic anhydrous | 45.6 |
| powder (J T Baker, 1430-01) | |
| lactose (Hormel CFI 14158) | 45.7 |
| Magnesium stearate (Alfa Aesar A16244) | 1.1 |
| Total | 100.0 |

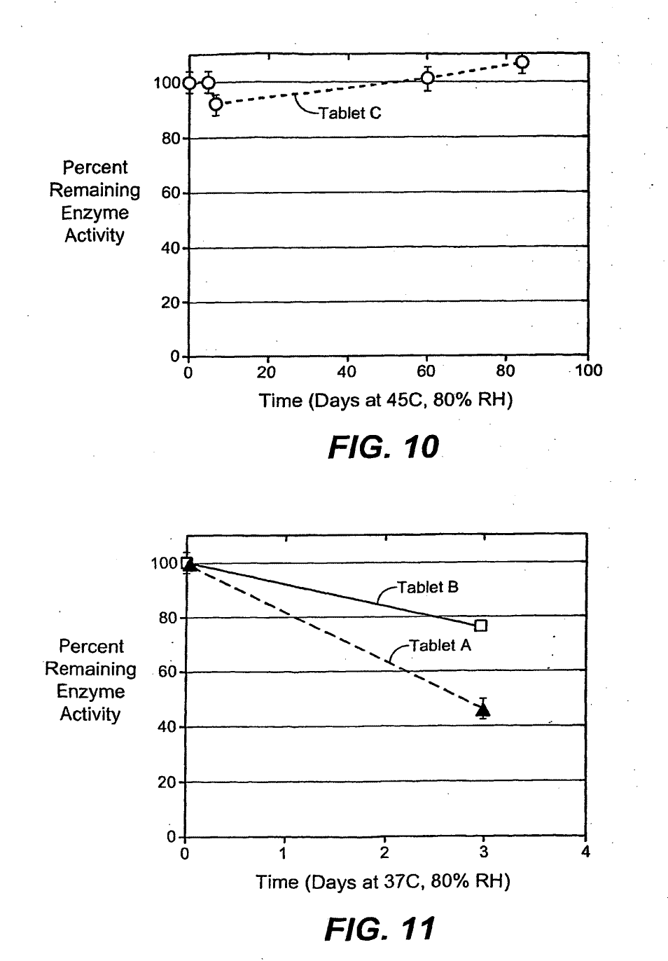

The tablets were then stored, unwrapped, under accelerated stability test conditions of 37° C. or 45° C. and 80% relative humidity. At various time intervals, tablets were removed from the storage environment, dissolved in a buffer, and measured for protease enzyme activity. The percent remaining protease activity, relative to the activity of the tablets at the start of the study was calculated, and is graphed in FIG. 10. FIG. 10 illustrates that the enzyme tablets have a high stability when stored at 80% relative humidity for more than 80 days. The percent remaining activity for the enzyme granules remains at least 100% over the storage period. The above stability study was conducted using two different control tablets that included bleaching agents and bleach activators along with the enzyme. The composition of the two control tablets is shown below in Tables 27 and 28.

TABLE 27
Composition of Enzyme and Bleaching Components Tablet A

| Ingredient | % (w/w) |
|---|---|
| Protease granules | 1 |
| Tripolyphosphate | 43 |
| Sodium silicate/Sodium carbonate mix (1:1 ratio) | 40 |
| TAED | 2 |
| Sodium percarbonate | 14 |
| Total | 100.0 |

TABLE 28
Composition of Enzyme and Bleaching Components Tablet B

| Ingredient | % (w/w) |
|---|---|
| Protease granules | 1 |
| Tripolyphosphate | 61 |
| Sodium carbonate | 16 |
| Sodium percarbonate | 16 |
| Sodium disilicate | 4 |
| TAED | 3 |
| Total | 100.0 |

FIG. 11 shows the percent remaining protease activity of tablets with bleach containing systems, when stored under the accelerated conditions described above. FIG. 11 demonstrates that bleach containing enzyme tablets made without enzyme stabilizers retain less than 100% enzyme activity following 3 days storage at 80% relative humidity. The bleach containing tablets were stored at only 37° C. and 80% relative humidity would have even less percent retained enzyme activity at a higher storage temperature of 45° C.

What is claimed is:

1. A enzyme tablet for providing a cleaning benefit as a single-dose additive to the wash cycle of an automatic laundry or dish washer, the wash cycle having a laundry or dish detergent therein; the enzyme tablet comprising an enzyme.

2. The enzyme tablet of claim 1 wherein the cleaning benefit is a decreased temperature of a wash cycle, a decreased time of a wash cycle, or a decreased temperature and time of a wash cycle.

3. The enzyme tablet of claim 1 with the proviso that the tablet is substantially without surfactants and bleaching agents and the cleaning benefit is selected from a decreased temperature of a wash cycle, a decreased time of a wash cycle, increased wash performance, and combinations thereof.

4. The enzyme tablet of claim 1 further comprising a binder or filler, a lubricant, and a disintegrant.

5. The enzyme tablet of claim 3 further comprising a binder or filler, a lubricant, and a disintegrant.

6. The enzyme tablet of claim 1 wherein the enzyme has a dosage selected to provide about 0.1 ppm to about 50 ppm enzyme to the wash cycle of the automatic laundry or dish washer.

7. The enzyme tablet of claim 3 wherein the enzyme has a dosage selected to provide about 0.1 ppm to about 50 ppm enzyme to the wash cycle of the automatic laundry or dish washer.

8. The enzyme tablet of claim 1 having a diameter of about 14 mm to about 36 mm.

9. The enzyme tablet of claim 1 wherein the enzyme is selected from protease, amylase, cellulase, lipase, phospholipase, cutinase, oxidase, oxygenase, transferase, reductase, hemicellulase, mannanase, esterase, isomerase, pectinase, lactase, peroxidase, lyase, laccase, and combinations thereof

10. The enzyme tablet of claim 2 wherein the increased wash performance is an equal or increased delta reflectance (L*), or delta reflectance (Y*), or delta color (E*) chromaticity value.

11. The enzyme tablet of claim 3 wherein the increased wash performance is an increased delta reflectance (L*), or delta reflectance (Y*), or delta color (E*) chromaticity value.

12. The enzyme tablet of claim 10 wherein the equal or increased delta reflectance (L*), delta reflectance (Y*), or delta color (E*) chromaticity value is at least 0% to 2500% higher than delta reflectance (L*), or delta reflectance (Y*), or delta color (E*) chromaticity values obtained without the addition of the enzyme tablet to the wash cycle.

13. The enzyme tablet of claim 11 wherein the increased delta reflectance (L*), delta reflectance (Y*), or delta color (E*) chromaticity value is at least 1% to 2500% higher than delta reflectance (L*), or delta reflectance (Y*), or delta color (E*) chromaticity values obtained without the addition of the enzyme tablet to the wash cycle.

14. The enzyme tablet of claim 2 wherein the decreased temperature of a wash cycle is a wash cycle temperature at least 30%, at least 40%, at least 50% lower than a conventional wash cycle temperature of a laundry or dish washer.

15. The enzyme tablet of claim 14 wherein the decreased temperature is about 20° C. lower than the conventional wash cycle temperature.

16. The enzyme tablet of claim 15 wherein the decreased temperature is 24° C. to 38° C.

17. The enzyme tablet of claim 2 wherein the decreased time of a wash cycle is at least 50%, at least 60%, at least 70% shorter than a time of a conventional wash cycle of a laundry or dish washer.

18. The enzyme tablet of claim 17 wherein the decreased time is about 65 minutes to about 105 minutes shorter than the conventional wash cycle of a European laundry or dish washer.

19. The enzyme tablet of claim 18 wherein the decreased time is 32 minutes.

20. The enzyme tablet of claim 1 having an enzyme stability of at least 80%, at least 90%, at least 100% after storage for at least 3 days and up to at least 80 days at 45° C. and 80% humidity.

21. An enzyme kit for consumers, the kit comprising at least one tablet containing at least one enzyme, the tablet enclosed in packaging material, the at least one tablet providing a cleaning benefit as a single-dose additive to an automatic laundry or dish washing cycle having a conventional laundry or dish detergent.

22. (canceled)

23. The enzyme kit of claim 21 wherein the at least one enzyme is selected from protease, amylase, cellulase, lipase, phospholipase, cutinase, oxidase, oxygenase, transferase, reductase, hemicellulase, mannanase, esterase, isomerase, pectinase, lactase, peroxidase, lyase, laccase, and combinations thereof.

24-34. (canceled)

35. A method for providing enzymes to consumers for boosting the wash performance of a conventional laundry or dish detergent in a laundry or dish washer; the method comprising: selecting at least one enzyme for laundry or dish detergent applications; combining the at least one enzyme with at least binders or fillers; manufacturing tablets from the combined enzyme and binders or fillers; and packaging the tablets for sale to consumers with instructions for use.

36-39. (canceled)

40. A method for providing a cleaning benefit in a wash cycle in laundry and dish washing applications, the method comprising: selecting on a laundry or dish washing machine a decreased temperature and/or a decreased time for a wash cycle; adding a laundry or dish washing compound and an enzyme tablet to the selected wash cycle.

41. An enzyme tablet additive to the wash cycle of an automatic laundry or dish washer, the wash cycle having a laundry or dish detergent therein; the enzyme tablet a single-dose additive comprising an enzyme, with the proviso that the tablet is substantially without surfactants and bleaching agents; the enzyme tablet enabling reduction of a time of a European wash cycle by 50% and/or reduction of a temperature of the wash cycle by at least 30%.
